(19) United States
(12) Patent Application Publication
(10) Pub. No.: US 2006/0002889 A1
(43) Pub. Date: Jan. 5, 2006

(54) Polyionene polymers with hydrolyzable linkages

(75) Inventor: Richard J. Fitzpatrick, Marblehead, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Genzyme Corporation, Cambridge, MA (US)

(21) Appl. No.: 11/132,156

(22) Filed: May 18, 2005

Related U.S. Application Data
(63) Continuation of application No. PCT/US03/36927, filed on Nov. 19, 2003.
(60) Provisional application No. 60/427,517, filed on Nov. 19, 2002.

(51) Int. Cl. A61K 31/785; C08L 61/20

(52) U.S. Cl. 424/78.3; 525/428

(57) ABSTRACT

Ionene polymers are effective antipathogenic agents and can be used as a surface treatment or as a pharmaceutical agent. Ionene polymers typically are persistent in an environment because they degrade slowly, however, it is sometimes desirable to have a polymer that degrades over time. Ionene polymers that have one or more hydrolyzable groups in the polymer backbone represent a type of ionene polymer that degrades with time, and the size of the resulting polymer fragments can be controlled by the placement of the hydrolyzable groups.

### RELATED APPLICATION

This application is a continuation of International Application No. PCT/US03/36927, which designated the United States, was filed on Nov. 19, 2003, and was published in English, which claims the benefit of U.S. Provisional Application No. 60/427,517, filed on Nov. 19, 2002. The entire teachings of the International Application and U.S. Provisional Application are hereby incorporated herein by reference.

### BACKGROUND OF THE INVENTION

Infectious entities such as bacteria, fungi, algae, viruses, protozoa, and the like are capable of growing on a wide variety of living and non-living surfaces, including skin, teeth, mucosa, vascular tissue, medical implants, and medical devices. Invasive viral, parasitic and microbial (e.g. bacterial, protozoal, fungal, etc.) infections of living organisms can affect various organs of the body. Such infections have historically been treated with well-characterized antipathogenic agents. However, the resistance of microorganisms to various antimicrobial agents has increased at an alarming rate, rendering many important therapeutics for the treatment of microbial infections ineffective.

When planktonic microorganisms grow and disseminate on non-living surfaces, they may cause contamination and biofouling of that surface. In many cases a microorganism can grow and accumulate on a surface to the point of becoming almost impossible to remove. This accumulation takes place through the formation of biofilms. Microbial contamination and biofilms adversely affect the health care industry and other industries wherein microbial contamination poses a health risk to humans such as public water supplies, and food production facilities.

Polyionenes have been recognized as a new type of antipathogenic compound to which pathogens often only slowly develop resistance. Polyionenes are safe to administer to warm-blooded animals and can also be used to coat surfaces, including surfaces of implantable devices.

Although polyionenes are effective antipathogenics, they degrade very slowly. For example, they will tend to persist in the lungs when administered by inhalation to treat pulmonary infections, particularly in patients with poor lung clearance. In addition, they will tend to accumulate when used to disinfect surfaces and must be removed by an additional washing step. It would be advantageous to develop ionenes that can be more readily cleared from organs such as the lungs or from surfaces after use.

Thus there is a need for antipathogenic agents that are non-toxic, degradable and effective at controlling contamination and infection by unwanted microbial organisms, with minimal development of resistant or polyresistant microorganisms.

### SUMMARY OF THE INVENTION

The present invention includes a variety of ionene polymers and oligomers (collectively referred to as polymers for ease of reference) comprising hydrolyzable groups in the polymer backbone. In one aspect, the ionene polymers contain tertiary or quaternary phosphorus or quaternary nitrogen atoms.

In one embodiment, the present invention is a hydrolyzable ionene copolymer comprised of repeat units represented by Structural Formulas (I) and (II): 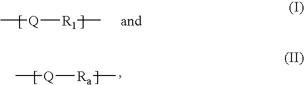
where:
each R₁ is a linker;
each Rₐ is independently a hydrolyzable group or a substituted or unsubstituted hydrocarbyl group interrupted with one or more hydrolyzable groups;
each Q is independently: 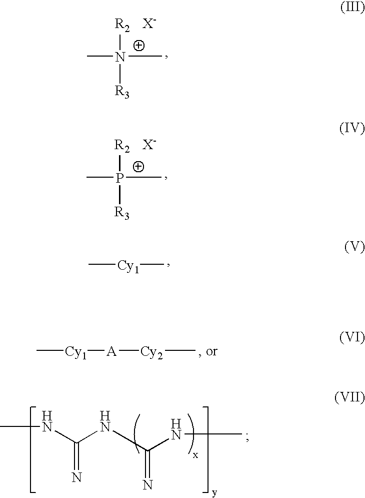
   Cy₁ and Cy₂ are each independently a quaternary nitrogen-containing monocyclic heteroaromatic ring, a tertiary nitrogen-containing non-aromatic ring or a quaternary nitrogen-containing non-aromatic heterocyclic ring;
   A is a covalent bond, or a substituted or unsubstituted lower alkylene group;
   R₂ and R₃ are independently —H or a substituted or unsubstituted aliphatic or aromatic group, such as an alkyl group or a hydroxyalkyl group;
   each X⁻, separately or taken together with other X⁻s, is a physiologically acceptable anion;
   x is an integer from 0-4; and
   y is an integer from 1-5.

Preferably, Q of Structural Formula (I) and Q of Structural Formula (II) are the same in copolymers of the present invention. Even more preferably, Q of Structural Formula (I) and Structural Formula (II) are each represented by Structural Formula (VI).

In another embodiment, the present invention is a hydrolyzable ionene homopolymer or copolymer comprising a repeat unit represented by Structural Formula (II). In ionene homopolymers and copolymers containing Cy₁ and Cy₂, Cy₁ and Cy₂ are each typically independently a quaternary nitrogen-containing monocyclic heteroaromatic ring or a quaternary nitrogen-containing non-aromatic heterocyclic ring. Other variables are as described above.

The present invention also includes a pharmaceutical composition comprising a carrier or diluent and a hydrolyzable ionene homopolymer or copolymer, as described herein.

In addition, the present invention is directed to a method of inhibiting colonization by a virus, parasite or microbe (i.e., the presence of a virus, parasite or microbe) or treating a viral, parasitic or microbial infection in a mammal comprising the step of administering to said mammal an effective amount of an ionene polymer comprising hydrolyzable groups in the polymer backbone (e.g., a copolymer comprised of repeat units represented by Structural Formula (I) and (II) or a homopolymer or copolymer comprised of repeat units represented by Structural Formula (II)).

Other methods encompassed by the present invention include a method of inhibiting the colonization of a surface by a virus, parasite or microorganism or the growth of a virus, parasite or microorganism on a surface comprising the step of contacting the surface with an effective amount of a polymer disclosed herein, a method of treating mucositis in a mammal comprising the step of administering to the mammal an effective amount of a polymer disclosed herein, and a method of preventing or inhibiting colonization or preventing or treating infection in a cystic fibrosis patient comprising the step of administering to the patient an effective amount of a polymer disclosed herein.

Polymers described herein have the advantage that they degrade over time. The degradation rate of an ionene polymer can be, in part, controlled by including a hydrolyzable subunit within the backbone. For example, the number of hydrolyzable subunits can be varied to alternatively hasten or delay degradation. In addition, the number and location of the hydrolyzable units will determine the size of the degradation products. The hydrolyzable ionene polymers described herein are expected to provide antiviral and antimicrobial activity similar to non-hydrolyzable ionene polymers, however, the present polymers are particularly appropriate when a finite lifetime is desired. Thus, they are particularly suitable for administering directly into the lungs for treating microbial infections therein, for example, microbial infections associated with cystic fibrosis.

### DETAILED DESCRIPTION OF THE INVENTION

The present invention discloses a plurality of ionene polymers having one or more repeat units with a hydrolyzable moiety. Hydrolyzable moieties are defined as functional groups that can be cleaved by water to give a rate of hydrolysis for a suitable half-life as described below, and include moieties that are bioerodable (e.g., can be cleaved by enzymes). Suitable hydrolyzable groups include esters, carbonates, carbamates, orthoesters, orthocarbonates, acetals, ketals, phosphazenes, carboxyacetals, carboxyorthoesters, thioorthoesters, sulfoxyorthoesters, and alpha-hydroxy acids. Preferably, all of the hydrolyzable groups contained in a repeat unit or a polymer are the same (e.g., all esters, all carbonates, all carbamates).

Typically, the rate of hydrolysis is accelerated in the presence of an acid, a base, a metal ion catalyst and/or an enzyme. The rate of hydrolysis can be further modulated by the type of substituents in the vicinity of the hydrolyzable group, for example, electron-withdrawing substituents increase the rate of hydrolysis and electron-donating substituents decrease the rate of hydrolysis. The rate of hydrolysis of polyionenes (as measured in water, an aqueous solution, in vitro or in vivo) disclosed herein typically results in a half-life on the order of minutes, days or weeks, but an appropriate rate of hydrolysis is selected according to the desired use for the hydrolyzable polyionene (e.g., to give a suitable rate of clearance from the lungs). A suitable rate of hydrolysis can be a rate that gives a half-life of less than about one year, less than about 6 months, less than about 3 months, less than about 2 months, less than about 1 month, less than about 3 weeks, less than about 2 weeks, less than about 1 week, less than about 100 hours, less than about 75 hours, less than about 50 hours, less than about 36 hours, less than about 24 hours, less than about 18 hours, less than about 12 hours, less than about 8 hours, less than about 6 hours, less than about 4 hours, less than about 2 hours or less than about one hour.

“Ionene polymers” or “polyionenes,” as used in the present invention, are cationic polymers or copolymers with quaternary nitrogen or phosphorus (e.g., having four carbons bonded to the nitrogen or phosphorus atom) or a protonated secondary or tertiary nitrogen or phosphorus located in the main polymeric chain or backbone of the polymer, providing a positive charge. The positive charge can be acquired in situ, such as in the case of a polymer containing tertiary nitrogen which becomes protonated in the presence of an acid, even a weak acid such as water. Polyionenes can also be polyguanidines or copolymers thereof, where the cationic nitrogen atom is an imide nitrogen directly bonded to the polymer backbone. Additional ionene polymer repeat units are disclosed in U.S. Ser. Nos. 60/262,586, filed Jan. 18, 2001, Ser. No. 10/051,765, filed Jan. 17, 2002, and Ser. No. 10/051,766, filed Jan. 17, 2002 (U.S. Publication Nos. 2003/0021761 A1 and 2003/0031644 A1), the contents of which are incorporated herein by reference.

The present invention provides the use of the hydrolyzable ionene polymers disclosed herein in the treatment of a disease or condition disclosed herein. In addition, the invention also provides the use of the hydrolyzable ionene polymers disclosed herein in the manufacture of a medicament for the treatment of a disease or condition disclosed herein.

The molecular weight of hydrolyzable ionene polymers is generally not limiting, however, molecular weights typically range from 500 to 500,000 Daltons, 500 to 100,000 Daltons, 500 to 20,000 Daltons, 500 to 10,000 Daltons, 500 to 5,000 Daltons, 1,000 to 5,000 Daltons, 500 to 3,000 Daltons, or 1,000 to 3,000 Daltons.

A linker group, as used herein, is a bond or a group that connects two ionene groups. A linker group should be inert and should not adversely affect the properties of the molecule, e.g., decrease activity or increase toxicity. Linker groups are typically hydrocarbylene groups such as substituted or unsubstituted arylene or (straight chained lower) alkylene groups. Hydrocarbylene groups can be interrupted by one or more heteroatoms, such as in a polyethylene glycol group. Suitable substituents for a hydrocarbylene group are described in the section providing alkyl group substituents. A common substituent for a linker group is a hydroxyl group.

Polyionenes suitable for use in the present invention comprises a repeat unit where Q is represented by Structural Formula (III) or Structrual Formula (IV). When Q is represented by Structural Formula (III), R₁ is preferably a substituted or unsubstituted phenylene, lower alkylene, polyalkylene glycol group, or —CH₂CHOH(CH₂)ₙCHOHCH₂—, where n is an integer ranging from 0 to 8, and R₂ and R₃ are as defined above. Even more preferably, R₁ is a substituted or unsubstituted straight chained lower alkylene group or polyalkylene glycol optionally substituted with one or more hydroxyl groups.

Examples of a copolymeric ionene repeat unit where Q is represented by Structural Formula (III) are represented by the formulas: 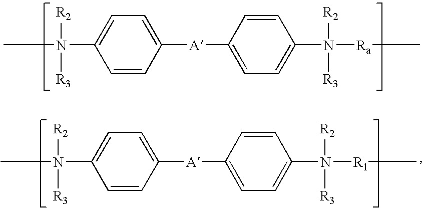
where Rₐ, R₁, R₂ and R₃ are as listed above and A′ is an alkylene or alkenylene group.

In one example of an ionene group, Q is represented by Structural Formula (V), where Cy₁ is a piperidinium ring having a tertiary nitrogen or a quaternary nitrogen additionally substituted with a substituted or unsubstituted lower alkyl group. Preferably, the quaternary nitrogen is substituted with a lower alkyl or hydroxy substituted lower alkyl group. An example of where Q is a “piperidinium” ionene is represented in Structural Formula (VIII): 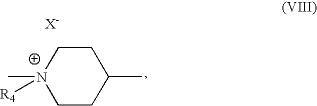
where R₄ is an electron pair, hydrogen or a substituted or unsubstituted lower alkyl group.

In one example of an ionene group where Q is represented by Structural Formula (VI), Cy₁ and Cy₂ are a piperidinium ring having a protonated tertiary nitrogen or a quaternary nitrogen. Each nitrogen can be independently substituted with a hydrogen (i.e., to give a protonated tertiary nitrogen) or a substituted or unsubstituted lower alkyl group (i.e., to give a quaternary nitrogen) and A is as defined above. More preferably, the quaternary nitrogen is substituted with a lower alkyl or hydroxy substituted lower alkyl group. An example of a “piperidinium” ionene repeat unit of this type is represented by Structural Formula (IX): 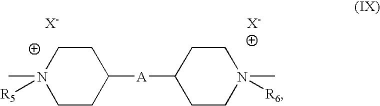
where A is as defined above, and R₅ and R₆ are each independently an electron pair, hydrogen or a substituted or unsubstituted lower alkyl group. Preferably, R₅ and R₆ are each independently an alkyl group or a hydroxyalkyl group, more preferably R₅ and R₆ are the same; and A is an unsubstituted straight chained lower alkylene group. Even more preferably, A is an unsubstituted straight chained lower alkylene group and R₁, when present, is a substituted or unsubstituted straight chained lower alkylene or polyalkylene glycol group optionally substituted with one or more hydroxyl groups, preferably an unsubstituted polyalkylene glycol or —CH₂CHOH(CH₂)ₙCHOHCH₂—, where n is an integer ranging from 0 to 8. Specific examples of “piperidinium” ionene repeat units are represented by the Structural Formulas (X), (XI), (XII), (XIII), (XIV) and (XV):

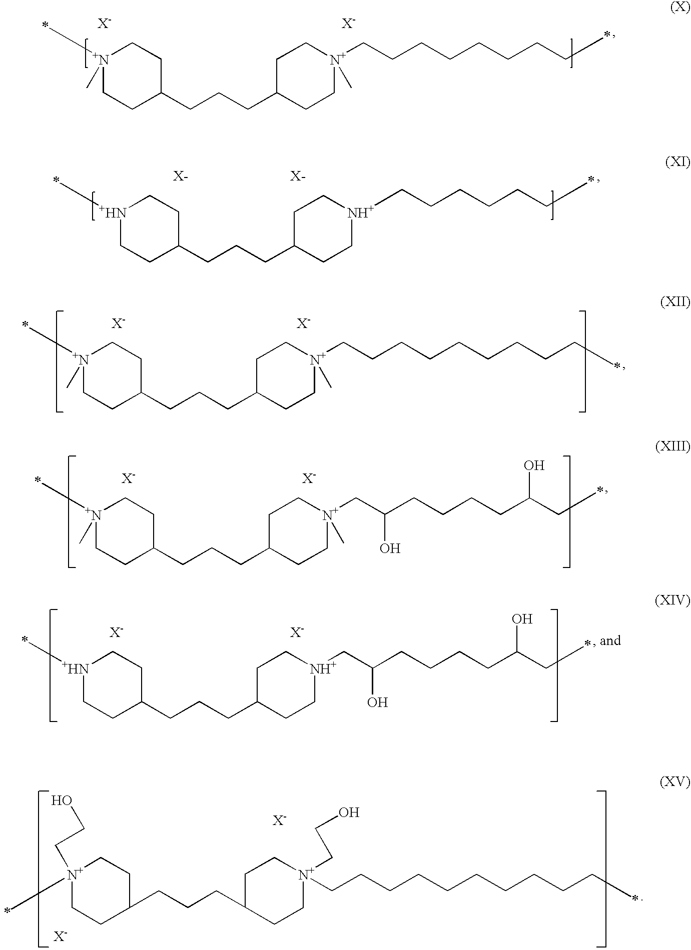

In a further example of an ionene group where Q is represented by Structural Formula (VI), Cy₁ and Cy₂ are each morpholine-type groups. In one example of a “morpholine” ionene polymer of this type, Q is represented by the structural formula: 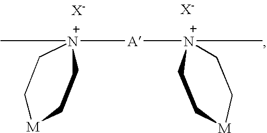
where A′ is or a substituted or unsubstituted alkylene or arylene group. M is (CH₂)ₘ, O, N or S, preferably N or O and more preferably O. More preferably, A is an unsubstituted alkylene group and M is O.

In another example of an ionene group where Q is represented by Structural Formula (VI), Cy₁ and Cy₂ are each pyridinium groups and A is as defined above. In one example of where Q is a “pyridinium” ionene group of this type, Q is characterized by Structural Formula (XVI): 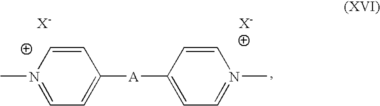
in which A and R₁ are as defined above. In a more preferred embodiment, A is an unsubstituted straight chained lower alkylene group. Even more preferably, A is an unsubstituted straight chained lower alkylene group and, when present, R₁ is a substituted or unsubstituted straight chained lower alkylene or polyalkylene glycol group optionally substituted with one or more hydroxyl groups, preferably an unsubstituted polyalkylene glycol or —CH₂CHOH(CH₂)ₙCHOHCH₂—, where n is an integer ranging from 0 to 8. Examples of preferred repeat units are represented by Structural Formulas (XVII) and (XVIII): 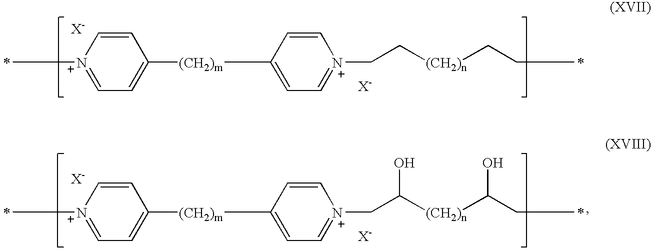
where m and n are independently-chosen integers ranging from 0 to 8. Typically, m is the same in each repeat unit and n is the same in each repeat unit.

Other specific examples of repeat units of polyionenes that can be used in the disclosed method are represented by Structural Formula (XVII) above, wherein m is 1 and n is 0; m is 1 and n is 1; m is 1 and n is 2; m is 1 and n is 4; m is 1 and n is 5; m is 1 and n is 6; m is 1 and n is 8; m is 2 and n is 0; m is 2 and n is 1; m is 2 and n is 2; m is 2 and n is 4; m is 2 and n is 5; m is 2 and n is 6; m is 2 and n is 8; m is 3 and n is 0; m is 3 and n is 1; m is 3 and n is 2; m is 3 and n is 4; m is 3 and n is 5; m is 3 and n is 6; m is 3 and n is 8; m is 4 and n is 0; m is 4 and n is 1; m is 4 and n is 2; m is 4 and n is 4; m is 4 and n is 5; m is 4 and n is 6; m is 4 and n is 8; m is 5 and n is 0; m is 5 and n is 1; m is 5 and n is 2; m is 5 and n is 4; m is 5 and n is 5; m is 5 and n is 6; and m is 5 and n is 8.

Other specific examples of repeat units of polyionenes that can be used in the disclosed method are represented by Structural Formula (XVIII) above, wherein m is 1 and n is 0; m is 1 and n is 1; m is 1 and n is 2; m is 1 and n is 4; m is 1 and n is 5; m is 1 and n is 6; m is 1 and n is 8; m is 2 and n is 0; m is 2 and n is 1; m is 2 and n is 2; m is 2 and n is 4; m is 2 and n is 5; m is 2 and n is 6; m is 2 and n is 8; m is 3 and n is 0; m is 3 and n is 1; m is 3 and n is 2; m is 3 and n is 4; m is 3 and n is 5; m is 3 and n is 6; m is 3 and n is 8; m is 4 and n is 0; m is 4 and n is 1; m is 4 and n is 2; m is 4 and n is 4; m is 4 and n is 5; m is 4 and n is 6; m is 4 and n is 8; m is 5 and n is 0; m is 5 and n is 1; m is 5 and n is 2; m is 5 and n is 4; m is 5 and n is 5; m is 5 and n is 6; and m is 5 and n is 8.

A further example of an ionene polymer represented by Structural Formula (XVI) is represented by Structural Formula (XVIIIa): 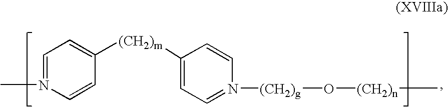
where m is an integer from 0 to 8, g is an integer from 1 to 6, and h is an integer from 1 to 6. In some embodiments of the invention, g and h can be different. Typically, g and h are each the same. Specific examples of repeat units of polyionenes represented by Structural Formula (XVIIIa) above include those where m is 1 and g and h are each 1; m is 1 and g and h are each 2; m is 1 and g and h are each 4; m is 1 and g and h are each 5; m is 1 and g and h are each 6; m is 2 and g and h are each 1; m is 2 and g and h are each 2; m is 2 and g and h are each 4; m is 2 and g and h are each 5; m is 2 and g and h are each 6; m is 3 and g and hare each 1; m is 3 and g and hare each 2; m is 3 and g and h are each 4; m is 3 and g and h are each 5; m is 3 and g and h are each 6; m is 4 and g and h are each 1; m is 4 and g and h are each 2; m is 4 and g and h are each 4; m is 4 and g and h are each 5; m is 4 and g and h are each 6; m is 5 and g and h are each 1; m is 5 and g and hare each 2; m is 5 and g and hare each 4; m is 5 and g and hare each 5; m is 5 and g and h are each 6; and m is 5 and g and h are each 8. One particular example of a repeat unit represented by Structural Formula (XVIIIa) is where m is 3 and g and h are each 4.

Another ionene polymer has repeat units represented by Structural Formula (XIX): 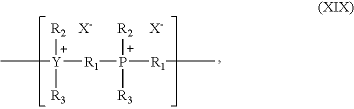
where Y is P or N, R₁ is a linker, and R₂ and R₃ are independently a substituted or unsubstituted aliphatic or aromatic group. R₁, R₂ and R₃ within formula (XIX) can be the same or different, but are preferably the same. Preferably, R₁ is a substituted or unsubstituted phenylene or lower alkylene group and R₂ and R₃ are independently a substituted or unsubstituted lower alkyl or phenyl group. More preferably, R₁ is an unsubstituted phenylene or lower alkylene group and R₂ and R₃ are independently an unsubstituted lower alkyl or phenyl group.

Examples of ionene repeat units represented by Structural Formula (XIX) include: 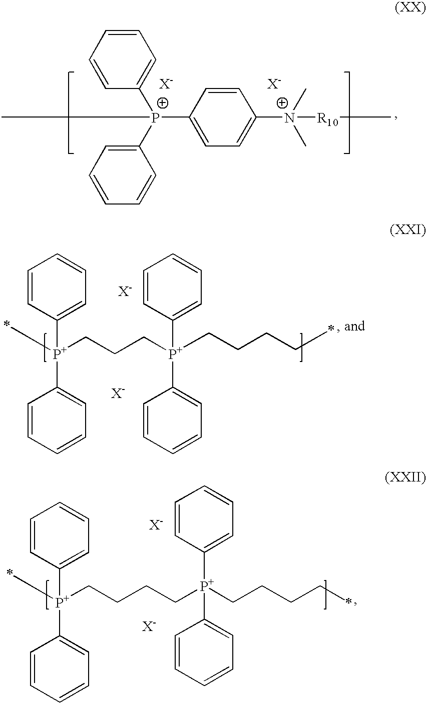
where R₁₀ is a substituted or unsubstituted lower alkylene group having from 4 to 12 carbon atoms and each X⁻, separately or taken together with other X⁻s is a physiologically acceptable anion.

In another embodiment of the present invention, the polyionene has repeat units where Q is represented by Structural Formula (VII). Preferably, R₁ is an unsubstituted lower alkylene or lower alkylene glycol group and x is 1 and y is 2; x is 1 and y is 3; x is 1 and y is 4; or x is 1 and y is 5. Specific examples of guanidine ionene polymers and copolymers comprise repeat units of formulas (XXIII), (XXIV), (XXV), (XXVI) and (XXVII):

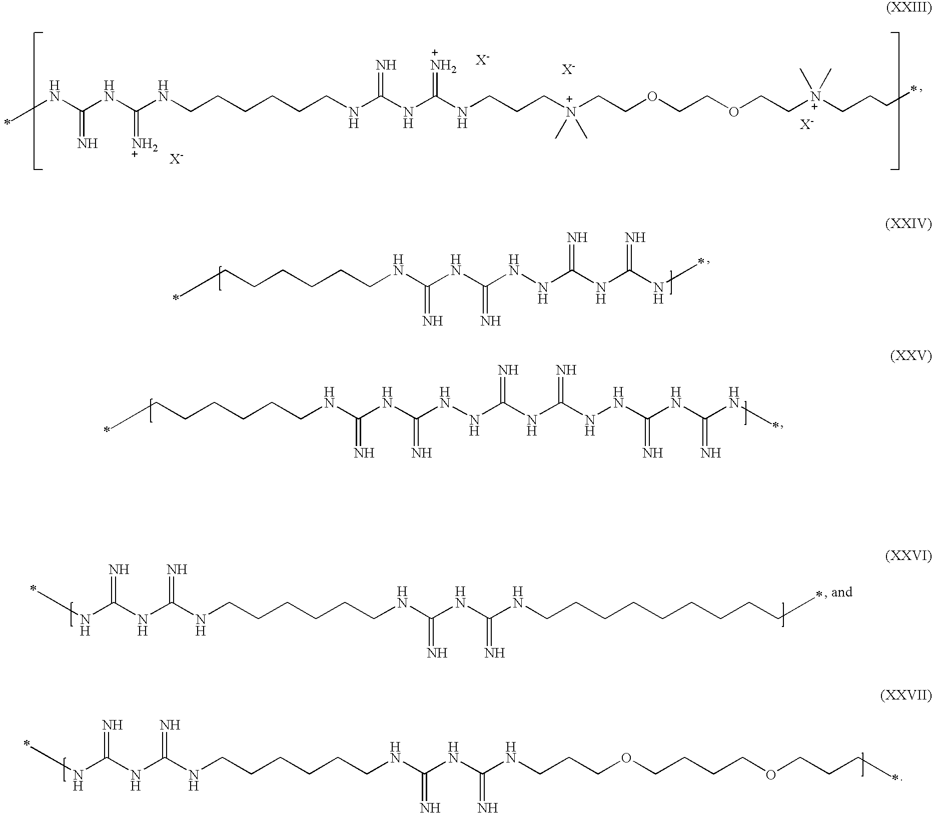

Examples of a repeat unit represented by Structural Formula (II) have the formulas: 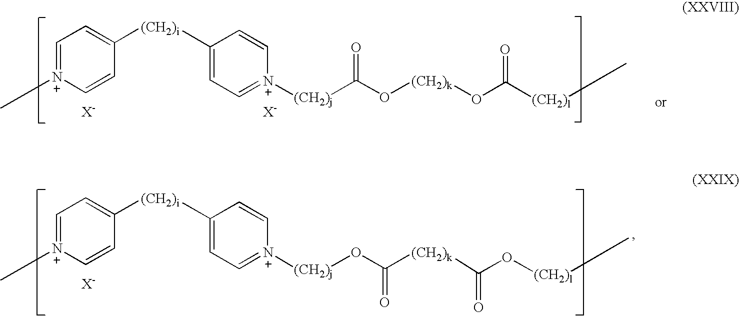
where i and j are integers independently ranging from 0 to 8 and k and l are integers independently ranging from 1 to 8, where k is preferably 1 to 3.

Additional examples of a repeat unit represented by Structural Formula (II) have the formulas: 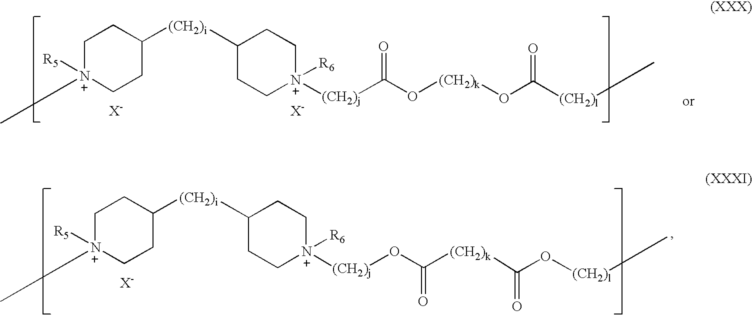
where R₅, R₆, i, j, k and l are as defined above, and R₅ and R₆ are preferably —H, methyl, ethyl, propyl or butyl and k is preferably 1 to 3.

Specific examples of repeat units represented by Structural Formulas (XXVIII), (XXIX), (XXX) and (XXXI) are chosen such that i is 0, j is 0, k is 1 and l is 1; i is 0, j is 0, k is 1 and l is 2; i is 0, j is 0, k is 1 and l is 3; i is 0, j is 0, k is 2 and l is 1; i is 0, j is 0, k is 2 and l is 2; i is 0, j is 0, k is 2 and l is 3; i is 0, j is 0, k is 3 and l is 1; i is 0, j is 0, k is 3 and l is 2; i is 0, j is 0, k is 3 and l is 3; i is 0, j is 1, k is 1 and l is 1; i is 0, j is 1, k is 1 and l is 2; i is 0, j is 1, k is 1 and l is 3; i is 0, j is 1, k is 2 and l is 1; i is 0, j is 1, k is 2 and l is 2; i is 0, j is 1, k is 2 and l is 3; i is 0, j is 1, k is 3 and l is 1; i is 0, j is 1, k is 3 and l is 2; i is 0, j is 1, k is 3 and l is 3; i is 0, j is 2, k is 1 and l is 1; i is 0, j is 2, k is 1 and l is 2; i is 0, j is 2, k is 1 and l is 3; i is 0, j is 2, k is 2 and l is 1; i is 0, j is 2, k is 2 and l is 2; i is 0, j is 2, k is 2 and l is 3; i is 0, j is 2, k is 3 and l is 1; i is 0, j is 2, k is 3 and l is 2; i is 0, j is 2, k is 3 and l is 3; i is 0, j is 3, k is 1 and l is 1; i is 0, j is 3, k is 1 and l is 2; i is 0, j is 3, k is 1 and l is 3; i is 0, j is 3, k is 2 and l is 1; i is 0, j is 3, k is 2 and l is 2; i is 0, j is 3, k is 2 and l is 3; i is 0, j is 3, k is 3 and l is 1; i is 0, j is 3, k is 3 and l is 2; i is 0, j is 3, k is 3 and l is 3; i is 1, j is 0, k is 1 and l is 1; i is 1, j is 0, k is 1 and l is 2; i is 1, j is 0, k is 1 and l is 3; i is 1, j is 0, k is 2 and l is 1; i is 1, j is 0, k is 2 and l is 2; i is 1, j is 0, k is 2 and l is 3; i is 1, j is 0, k is 3 and l is 1; i is 1, j is 0, k is 3 and l is 2; i is 1, j is 0, k is 3 and l is 3; i is 1, j is 1, k is 1 and l is 1; i is 1, j is 1, k is 1 and l is 2; i is 1, j is 1, k is 1 and l is 3; i is 1, j is 1, k is 2 and l is 1; i is 1, j is 1, k is 2 and l is 2; i is 1, j is 1, k is 2 and l is 3; i is 1, j is 1, k is 3 and l is 1; i is 1, j is 1, k is 3 and l is 2; i is 1, j is 1, k is 3 and l is 3; i is 1, j is 2, k is 1 and l is 1; i is 1, j is 2, k is 1 and l is 2; i is 1, j is 2, k is 1 and l is 3; i is 1, j is 2, k is 2 and l is 1; i is 1, j is 2, k is 2 and l is 2; i is 1, j is 2, k is 2 and l is 3; i is 1, j is 2, k is 3 and l is 1; i is 1, j is 2, k is 3 and l is 2; i is 1, j is 2, k is 3 and l is 3; i is 1, j is 3, k is 3 and l is 1; i is 1, j is 3, k is 1 and l is 2; i is 1, j is 3, k is 1 and l is 3; i is 1, j is 3, k is 2 and l is 1; i is 1, j is 3, k is 2 and l is 2; i is 1, j is 3, k is 2 and l is 3; i is 1, j is 3, k is 3 and l is 1; i is 1, j is 3, k is 3 and l is 2; i is 1, j is 3, k is 3 and l is 3; i is 2, j is 0, k is 1 and l is 1; i is 2, j is 0, k is 1 and l is 2; i is 2, j is 0, k is 1 and l is 3; i is 2, j is 0, k is 2 and l is 1; i is 2, j is 0, k is 2 and l is 2; i is 2, j is 0, k is 2 and l is 3; i is 2, j is 0, k is 3 and l is 1; i is 2, j is 0, k is 3 and l is 2; i is 2, j is 0, k is 3 and l is 3; i is 2, j is 1, k is 1 and l is 1; i is 2, j is 1, k is 1 and l is 2; i is 2, j is 1, k is 1 and l is 3; i is 2, j is 1, k is 2 and l is 1; i is 2, j is 1, k is 2 and l is 2; i is 2, j is 1, k is 2 and l is 3; i is 2, j is 1, k is 3 and l is 1; i is 2, j is 1, k is 3 and l is 2; i is 2, j is 1, k is 3 and l is 3; i is 2, j is 2, k is 1 and l is 1; i is 2, j is 2, k is 1 and l is 2; i is 2, j is 2, k is 1 and l is 3; i is 2, j is 2, k is 2 and l is 1; i is 2, j is 2, k is 2 and l is 2; i is 2, j is 2, k is 2 and l is 3; i is 2, j is 2, k is 3 and l is 1; i is 2, j is 2, k is 3 and l is 2; i is 2, j is 2, k is 3 and l is 3; i is 2, j is 3, k is 1 and l is 1; i is 2, j is 3, k is 1 and l is 2; i is 2, j is 3, k is 1 and l is 3; i is 2, j is 3, k is 2 and l is 1; i is 2, j is 3, k is 2 and l is 2; i is 2, j is 3, k is 2 and l is 3; i is 2, j is 3, k is 3 and l is 1; i is 2, j is 3, k is 3 and l is 2; i is 2, j is 3, k is 3 and l is 3; i is 3, j is 0, k is 1 and l is 1; i is 3, j is 0, k is 1 and l is 2; i is 3, j is 0, k is 1 and l is 3; i is 3, j is 0, k is 2 and l is 1; i is 3, j is 0, k is 2 and l is 2; i is 3, j is 0, k is 2 and l is 3; i is 3, j is 0, k is 3 and l is 1; i is 3, j is 0, k is 3 and l is 2; i is 3, j is 0, k is 3 and l is 3; i is 3, j is 1, k is 1 and l is 1; i is 3, j is 1, k is 1 and l is 2; i is 3, j is 1, k is 1 and l is 3; i is 3, j is 1, k is 2 and l is 1; i is 3, j is 1, k is 2 and l is 2; i is 3, j is 1, k is 2 and l is 3; i is 3, j is 1, k is 3 and l is 1; i is 3, j is 1, k is 3 and l is 2; i is 3, j is 1, k is 3 and l is 3; i is 3, j is 2, k is 1 and l is 1; i is 3, j is 2, k is 1 and l is 2; i is 3, j is 2, k is 1 and l is 3; i is 3, j is 2, k is 2 and l is 1; i is 3, j is 2, k is 2 and l is 2; i is 3, j is 2, k is 2 and l is 3; i is 3, j is 2, k is 3 and l is 1; i is 3, j is 2, k is 3 and l is 2; i is 3, j is 2, k is 3 and l is 3; i is 3, j is 3, k is 1 and l is 1; i is 3, j is 3, k is 1 and l is 2; i is 3, j is 3, k is 1 and l is 3; i is 3, j is 3, k is 2 and l is 1; i is 3, j is 3, k is 2 and l is 2; i is 3, j is 3, k is 2 and l is 3; i is 3, j is 3, k is 3 and l is 1; i is 3, j is 3, k is 3 and l is 2; and i is 3, j is 3, k is 3 and l is 3.

Alternatively, the hydrolyzable ester groups in Structural Formulas (XXVIII)-(XXXI) are replaced with —O—C(O)—O—. In another alternative, the hydrolyzable ester groups in Structural Formulas (XXVIII)-(XXXI) are replaced with —O—C(O)—NH—. In yet another alternative, the hydrolyzable ester groups of Structural Formulas (XXVIII)-(XXXI) are replaced with —NH—C(O)—O—.

Further examples of repeat units represented by Structural Formula (II) are represented by the following structures: 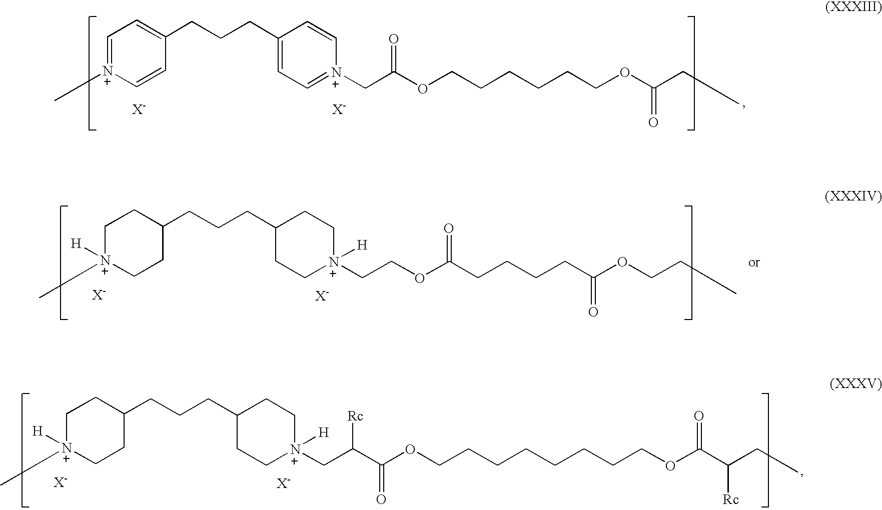
where R_{c} is —H or a substituted or unsubstituted alkyl group.

A number of properties of the copolymer can be adjusted based on the ratio of repeat units represented by Structural Formula (I) and (II). For example, the size of the degradation product can be decreased and/or the desired rate of degradation can be accelerated by increasing the number of hydrolyzable groups. Typically, the disclosed copolymers comprise 10-90% mole, 10-70% mole, or 10-50% mole of repeat units represented by Structural Formula (I). Similarly, the disclosed copolymers can comprise 10-90% mole, 30-90% mole, or 50-90% mole of repeat units represented by Structural Formula (II). Repeat units represented by Structural Formula (I) are typically, but not necessarily, identical throughout a polymer. The same is true for repeat units represented by Structural Formula (II), such as copolymers where all repeat units are represented by Structural Formula (II).

In further embodiments of the invention, a copolymer consists of repeat units represented by Structural Formula (I) and Structural Formula (II). For example, the molar ratio of repeat units represented by Structural Formula (I) and (II) in the disclosed polymers can be about 1:1, about 2:1, about 3:1, about 4:1, about 5:1, about 10:1, about 1:2, about 1:3, about 1:4, about 1:5 or about 1:10.

Alternatively, the proportion of hydrolyzable groups in a polyionene copolymer can be expressed in terms of the number of hydrolyzable groups per unit mass of the copolymer. Suitable ratios include one hydrolyzable group per 100 to 5,000 Daltons, per 100 to 3,000 Daltons, per 200 to 2,000 Daltons, per 200 to 1,000 Daltons or per 200 to 500 Daltons. Other suitable ratios for the number of hydrolyzable groups per unit mass of the copolymer include at least one hydrolyzable group per 100 Daltons, per 200 Daltons, per 500 Daltons, per 1000 Daltons, per 2000 Daltons, or per 3000 Daltons. Such relationships between the number of hydrolyzable groups per unit mass can also be applied to ionene homopolymers.

An “aliphatic group” is non-aromatic, consists solely of carbon and hydrogen and may optionally contain one or more units of unsaturation, e.g., double and/or triple bonds. An aliphatic group may be straight chained, branched, or cyclic and typically contains between about 1 and about 24 carbon atoms, more typically between about 1 and about 12 carbon atoms.

Aliphatic groups are preferably lower alkyl groups or lower alkylene or alkenylene groups, which include C1-24 (preferably C1-C12) straight chained or branched saturated hydrocarbons. An alkyl group is a saturated hydrocarbon in a molecule that is bonded to one other group in the molecule through a single covalent bond from one of its carbon atoms. Examples of lower alkyl groups include methyl, ethyl, n-propyl, iso-propyl, n-butyl, sec-butyl and tert-butyl. An alkylene group is a saturated hydrocarbon in a molecule that is bonded to two other groups in the molecule through single covalent bonds from two of its carbon atoms. Examples of lower alkylene groups include methylene, ethylene, propylene, iso-propylene (—CH(CH₂)CH₂—), butylene, sec-butylene (—CH(CH₃)CH₂CH₂—), and tert-butylene (—C(CH₃)₂CH₂—). An alkenylene group is similar to an alkyl group, but has one or more double bonds.

Aromatic groups include carbocyclic aromatic groups such as phenyl, 1-naphthyl, 2-naphthyl, 1-anthracyl and 2-anthacyl, and heterocyclic aromatic groups such as N-imidazolyl, 2-imidazolyl, 2-thienyl, 3-thienyl, 2-furanyl, 3-furanyl, 2-pyridyl, 3-pyridyl, 4-pyridyl, 2-pyrimidyl, 4-pyrimidyl, 2-pyranyl, 3-pyranyl, 3-pyrazolyl, 4-pyrazolyl, 5-pyrazolyl, 2-pyrazinyl, 2-thiazolyl, 4-thiazolyl, 5-thiazolyl, 2-oxazolyl, 4-oxazolyl and 5-oxazolyl.

Aromatic groups also include fused polycyclic aromatic ring systems in which a carbocyclic aromatic ring or heteroaryl ring is fused to one or more other heteroaryl rings. Examples include 2-benzothienyl, 3-benzothienyl, 2-benzofuranyl, 3-benzofuranyl, 2-indolyl, 3-indolyl, 2-quinolinyl, 3-quinolinyl, 2-benzothiazolyl, 2-benzooxazolyl, 2-benzimidazolyl, 2-quinolinyl, 3-quinolinyl, 1-isoquinolinyl, 3-quinolinyl, 1-isoindolyl and 3-isoindolyl. Phenyl is a preferred aromatic group.

“Arylene” is an aromatic ring(s) moiety in a molecule that is bonded to two other groups in the molecule through single covalent bonds from two of its ring atoms. Examples include phenylene —[—(C₆H₄)—], thienylene [—(C₄H₂S)—] and furanylene [—(C₄H₂O)—].

A nitrogen-containing non-aromatic heterocyclic group is a cyclic group containing one or more nitrogen atoms in the ring, which can have one or more degrees of unsaturation provided that the group is not aromatic. Examples of nitrogen-containing non-aromatic heterocyclic groups include aziridine, azetidine, pyrrolidine, 2,3-1H-pyrrole, piperidine, morpholine, thiomorpholine, 1,2,3,4-tetrahydropyridine and 1,4-dihydropyridine.

A polyalkylene glycol is an alkylene group, which includes one or more ether linkages, where the chain includes a total of about 1 to about 12 carbon and oxygen atoms, and is optionally substituted with one or more hydroxyl groups. Preferably, the polyalkylene glycol is polyethylene glycol or polypropylene glycol.

A “hydrocarbyl group” is an alkylene or arylene group, i.e., —(CH₂)ₙ— or —(CH₂)ₓC₆H₄(CH₂)ₓ—, where x is a positive integer (e.g., from 1 to about 30), preferably between 6 and about 30, more preferably between 6 and about 15. The carbon chain of the hydrocarbyl group may be optionally interrupted with one or more ether (—O—), thioether (—S—), amine [—N(R^{a})—] or ammonium [—N⁺(R^{a}R^{b})—] linkages, or a combination thereof. R^{a} and R^{b} are independently —H, alkyl, substituted alkyl, phenyl, or substituted phenyl. R^{a} and R^{b} can be the same or different, but are typically the same. Examples of hydrocarbyl groups include butylene, pentylene, hexylene, heptylene, octylene, nonylene, decylene, dodecylene, 4-oxaoctylene, 5-oxanonylene, 4-azaoctylene, 4-thiaoctylene, 3,6-dioxaoctylene, 3,6-diazaoctylene, and 4,9-dioxadodecane.

Examples of suitable substituents on a hydrocarbyl, aliphatic, aromatic or benzyl group may include, for example, halogen (—Br, —Cl, —I and —F), —OR, —CN, —NO₂, —NR₂, —COOR, —CONR₂, —SOₖR (k is 0, 1 or 2), and —NH—C(═NH)—NH₂. An aliphatic group can also have ═O or ═S as a substituent. Each R is independently —H, an aliphatic group, a substituted aliphatic group, a benzyl group, a substituted benzyl group, an aromatic group or a substituted aromatic group, and preferably —H, a lower alkyl group, a benzylic group or a phenyl group. Substituent groups can be selected such that all substituents are either neutral or positively charged. A substituted benzylic group or aromatic group can also have an aliphatic or substituted aliphatic group as a substituent. A substituted aliphatic group can also have a benzyl, substituted benzyl, aromatic or substituted aromatic group as a substituent. A substituted hydrocarbyl, aliphatic, substituted aromatic or substituted benzyl group can have more than one substituent. A preferred substituent on an aliphatic group is —OH.

The anions represented by X⁻ in the polymer can be the same or different. Each X⁻ in a repeat unit can separately be a monovalent anion, i.e., an anion having a negative charge of one. Alternatively, two or more X⁻s in the same repeat unit or in different repeat units, taken together, can represent an anion having a negative charge of two, three or more. A polymer can comprise anions of different charges. Examples of suitable counteranions include sulfate, bisulfate, sulfite, bisulfite, phosphate, monohydrogenphosphate, dihydrogenphosphate, metaphosphate, pyrophosphate, chloride, bromide, iodide, acetate, proprionate, decanoate, caprylate, acrylate, formate, isobutyrate, caproate, heptanoate, propiolate, oxalate, malonate, succinate, fumarate, maleate, benzoate, alkyl sulfonate, phenylacetate, citrate, lactate, glycolate, tartrate, carbonate, bicarbonate and the like.

One anion can be exchanged for a second anion by various methods described in U.S. Application No. 60/397,868 and PCT Application No. PCT/US03/22514, the contents of which are incorporated herein by reference. In one such method, a proportion of the first anions of the ionene polymer can be exchanged for another anion by dissolving the polyionene in a solution containing the second anion or a mixture of the first and second anions. Another anion exchange method involves contacting the polyionene with an anionic exchange resin loaded with the desired second anion. Ion exchange processes involving an anionic exchange resin can be carried out in a throw-away mode, a regenerative mode, or in a continuous counter-current mode in simulated moving bed (SMB) equipment. In a further method, a proportion of the first anions of the polyionene can be exchanged for a second anion by electrodialysis. In electrodialysis, for example, a polyionene solution and a solution containing a salt having a desired second anion are passed through alternate channels of a stack of cation and/or anion exchange membranes. Conditions such as voltage, current density, flow rate of the solutions, and operation in co- or counter-current mode are controlled to produce a polyionene with the desired anion content. Polyionenes that have had their anions altered by any of the previously described methods can be purified by ultrafiltering the polyionene. Typically, ultrafiltration occurs simultaneously with or following anion exchange. For processes involving electrodialysis, ultrafiltration typically occurs prior to electrodialysis. Ultrafiltering a polyionene typically includes one or more cycles of diluting and concentrating the polyionene, whereby anions not bound to the polyionene and other contaminants are forced through a membrane and removed during concentration.

Also included in the present invention are physiologically acceptable salts of the polymers having guanidine repeat units and non-quaternary amine groups. Salts can be formed by reacting the polymer with a suitable acid. Examples include the corresponding acid of the counteranions listed above. Polymers with guanidine repeat units can have up to one molecule of an acid such as hydrochloride or hydrobromide for every —NHC(═NH)NH— group in the repeat unit.

Ionene polymers of the invention and pharmaceutical compositions thereof provide numerous advantages over conventional therapies for treatment of microbial infections. As used herein, “conventional antimicrobial” therapies include but are not limited to well known antibacterial agents, such as vancomycin, metronidazole, penicillin, oxacillin, streptomycin, rifamycin, amphotericin B, griseofulvin, penicillin, cephalothin, cefazolin, chloramphenicol, fluconazole, clindamycin, erythromycin, bacitracin, vancomycin, ciprofloxiacin, tertracycline, and fusidic acid, as well as antifungals, antiseptics and the like. Ionene polymers of the invention provide a broader spectrum of treatment than presently available antibiotics. Ionene polymers are less likely to elicit antibiotic resistance or polyresistance. When desirable, ionene polymers of the invention may be designed such that they are not likely to be systemically absorbed by the body thus providing an attractive drug safety profile (e.g., sufficiently large size to exclude absorption by diffusion or pinocytosis or sufficient charge density to prevent diffusion across cell membranes). Ionene polymers of the present inventions can also be designed to be delayed-release polymers (e.g., when a subunit of the polymer has a therapeutic activity and/or when a subunit is more readily absorbed or taken up into a desired area).

Effective amounts of an ionene polymer to be administered will be determined on an individual basis, and will be determined at least in part, by consideration of the individual's size, the severity of symptoms to be treated and the result sought. As used herein, an effective amount refers to an appropriate amount of active ingredient (ionene polymer) to obtain therapeutic or prophylactic effect and can be determined by standard pharmaceutical procedures in cell cultures or experimental animals. Typical dosages range from between about 0.05 μg/kg body weight to about 500 mg/kg body weight, more typically between about 0.1 μg/kg body weight to about 100 mg/kg body weight and even more typically even more typically between about 0.5 μg/kg body weight and about 10 mg/kg body weight.

The polymer can be administered alone or in a pharmaceutical composition comprising the polymer, a pharmaceutically acceptable carrier, and optionally, one or more additional drugs. The polymers can be administered, for example, topically, ophthalmically, vaginally, orally, buccally, intranasally, by aerosol, rectally, by injection (e.g., intramuscular, intraperitoneal, subcutaneous) or by pulmonary means. Polymers of the present invention are advantageously administered to an epithelial surface in an organism. The form in which the polymer is administered, for example, powder, tablet, capsule, solution, or emulsion, depends in part on the route by which it is administered. Suitable carriers and diluents will be immediately apparent to persons skilled in the art. These carrier and diluent materials, either inorganic or organic in nature, include, for example, gelatin, albumin, lactose, starch, magnesium stearate preservatives (stabilizers), melting agents, emulsifying agents, salts and buffers. For topical administration, examples of pharmaceutically acceptable carriers include, for example, commercially available inert gels, or liquids supplemented with albumin, methyl cellulose or a collagen matrix. Typical of such formulations are ointments, creams and gels. The effective amount can be administered in a series of doses separated by appropriate time intervals such as minutes or hours.

Pathogenic infections which can be treated or prevented or inhibited (e.g., by preventing or inhibiting colonization) by administering an effective amount of an ionene polymer or a pharmaceutical composition thereof to a mammal infected with a microbe include, but are not limited to, bacterial infections, such as infection by species of Streptococcus, Salmonella, Campylobacter, Helicobacter, Burkholderia, Actinomyces, Eschericha, Mycobacteria, Pasturella, Francisella, Clostridium, Staphylococcus, Shigella, Pseudomonas, Listeria, Bacillus, Eikenella, Actinobacillus, Bacteriodes, Capnocytophaga, Wolinella, Bacteriodes, Mycoplasma, Treponema, Peptostreptococcus, Bacteriodes, Fusobacteria, Selenomonas, Bacteriodes, and Enterobacter. Specific species include Eschericha coli, Clostridium difficile, Eikenella corrodens, Actinobacillus actinomycetemcomitans, Bacteriodes gingivalis, Wolinella recta, Bacteriodes intermedius, Peptostreptococcus micros, Bacteriodes forsythus, Selenomonas sputigena, Bacteriodes fragilis, and Enterobacter cloacae. Other pathogenic infections include viral infections, protozoal infections, mycoplasma infections, fungal infections, and parasitic infections. The growth of these pathogens on a surface or the colonization of a surface can be inhibited by methods described in detail below.

In one preferred embodiment, polymers are administered to the oral cavity for treatment of infections and ulcers of the mouth. In another preferred embodiment, polymers of the invention are administered orally for treatment of pathogenic infections in the gastrointestinal tract of a mammal. In yet another preferred embodiment, polymers of the invention are administered topically for treatment of ocular pathogenic infections or for treatment of pathogenic infections on the skin of a mammal. One example of treatment of infections on the skin of a mammal is a wound management regimen that includes a polymer of the invention alone or in combination with a tissue sealant or other wound repair product as is known in the art.

One type of condition that can be advantageously treated with the disclosed polymers is mucositis. Mucositis is defined herein as inflammation and/or ulceration of a mucous membrane, which can be caused by infection, abrasion, radiation injury, chemical injury, antineoplastics, antibodies or other tissue injury. The disclosed method can be used to treat mucositis in the stomach, intestines, and the like; however, it is particularly effective when used to treat oral mucositis. Oral mucositis is characterized by inflammation of a mucous membrane of the oral cavity or lips and is typically accompanied by redness, swelling, and/or ulcerations of the mouth. Included in this description is oral mucositis that is a side-effect of anti-cancer therapies such as chemotherapy and radiotherapy, and oral mucositis that is a side effect of bone marrow transplantation or stem cell transplant or ablation. Treatment with an ionene polymer can be particularly beneficial for patients undergoing treatment for tumors of the head and neck, such as radiation patients. Mucositis also includes mucositis that develops spontaneously in a healthy patient not receiving anti-cancer therapy, as in the case of a canker sore or mouth ulcer.

Treatment of mucositis includes both prophylactic and therapeutic uses of the ionene polymers. Desired prophylactic effects include prevention of and inhibition of mucositis, reduction in severity of mucositis, reduction in size of mucositis lesions compared with, for example, what is normally experienced by a mammal undergoing cancer therapy, and reduction in likelihood of developing mucositis. Desired therapeutic effects include amelioration of the discomfort associated with the oral mucositis, and/or increased rate of healing of mucositis lesions compared with, for example, what is normally experienced by a mammal undergoing cancer therapy. Thus, the invention provides, in one aspect, a method of treating mucositis or oral mucositis comprising administering an effective amount of an ionene oligomer.

For prophylactic treatment of mucositis resulting from chemotherapy, treatment with an ionene oligomer is initiated before the onset of the chemotherapy, during chemotherapy, after chemotherapy is complete but before symptoms appear or any combination of the above. For prophylactic treatment of mucositis resulting from radiation therapy, treatment with the ionene polymer is initiated before the onset of radiation therapy, during radiation exposure, after radiation exposure has been terminated (preferably no sooner than about one hour, more preferably about five hours after termination) but before symptoms appear or treatment can begin in any combination of the above time periods. For therapeutic treatment of mucositis resulting from radiation therapy or chemotherapy, the ionene polymer is administered after symptoms of mucositis (e.g., mouth ulcers) have appeared.

The polymer for mucositis therapy can be administered alone or in a pharmaceutical composition comprising the polymer, a pharmaceutically acceptable carrier, and optionally, one or more additional drugs, e.g., antibiotics or antimicrobials. Examples include streptomycin, rifamycin, amphotericin B, griseofulvin, penicillin, cephalothin, cefazolin, chloramphenicol, fluconazole, clindamycin, erythromycin, bacitracin, vancomycin, ciprofloxiacin, tetracycline, and fusidic acid.

The polymers for mucositis therapy can be administered, for example, topically, orally, buccally, intranasally, by aerosol, rectally or vaginally, depending upon the site of the inflamed tissue or ulcer. The form in which the polymer is administered, for example, powder, tablet, capsule, solution, or emulsion, depends in part on the route by which it is administered. For oral mucositis, the polymer is preferably administered orally as a gargle, an ointment, a swab, a gel, and the like.

Suitable carriers and diluents for an ionene polymer used to treat mucositis will be immediately apparent to persons skilled in the art. These carrier and diluent materials, either organic or inorganic in nature, include, for example, gelatin, lactose, starch, magnesium stearate, preservatives (stabilizers), sugars, emulsifying agents, salts and buffers. When applied directly to the lesion, examples of pharmaceutically acceptable carriers include, for example, commercially available inert gels, or liquids supplemented with albumin, methyl cellulose, or a collagen matrix.

An effective amount of an ionene polymer to treat mucositis to be administered will be determined on an individual basis, and will be determined at least in part, by consideration of the individual's size, the severity of symptoms to be treated and the result sought. As used herein, an effective amount refers to an appropriate amount of ionene polymer, which results in a desired therapeutic or prophylactic effect with respect to mucositis, as defined above. Typical dosages for applied and/or ingested ionene polymers range from between about 0.05 μg/kg body weight to about 500 mg/kg body weight, more typically between about 0.1 μg/kg body weight to about 100 mg/kg body weight and even more typically between about 0.5 μg/kg body weight and about 10 mg/kg body weight.

The method of treating mucositis is preferably used with human patients, but can also be used with other mammals, such as companion animals (e.g., dogs, cats, and the like), farm animals (horses, cattle, goats, and the like) and laboratory animals (hamsters, mice, rats, and the like).

In another preferred embodiment, antimicrobial polymers of the invention are administered by inhalation for treatment or prevention or inhibition of pulmonary infections. Pulmonary infections suitable for treatment with polymers disclosed herein include pneumonia, bronchopneumonia and bronchitis, which are caused by pathogens including Streptococcus pneumoniae, Streptococcus pyogenes, Staphylococcus aureus, Legionella spp. (L. pneumophila, L. micdadei), Chlamydia pneumoniae, Chlamydia psittaci, Hemophilus influenzae, Enterobacteriaceae, Pseudomonas aeruginosa, Escherichia coli, Fusobacterium nucleatum, Bacteroides melaninogenicus, Bacteroides fragilis, Mycobacterium tuberculosis, Bacillus anthracis, Yersinia pestis, Francisella tularensis, Coxiella burnetti, Enterobacter spp., Proteus spp., Klebsiella spp., Brucella spp., Mycoplasma spp., Blastomyces spp., Leptospira spp., Histoplasma spp., Coccidioides spp., Cryptococcus spp., Candida spp., Pneumocystis carinii, Entamoeba histolytica, the influenza A and B viruses, the measles virus, cytomegalovirus and adenovirus.

Particular pulmonary infections or colonizations for which the disclosed polymers are effective include those which accompany cystic fibrosis. Patients suffering from cystic fibrosis (CF) produce excessive quantities of sweat and mucus. The mucus secreted is very thick and blocks passageways in the lungs and sinuses, causing them to be susceptible to colonization and/or infection by microbes and/or pathogens. Respiratory tract or pulmonary infections, which lead to respiratory inflammation and respiratory failure, are a primary cause of morbidity and mortality in CF patients. As there are currently no treatments to cure the root cause of CF (a defective protein, cystic fibrosis transmembrane conductance regulator), treatment of a patient suffering from CF with an ionene polymer represents a means of addressing the complications associated with CF. Present therapies for CF-associated infections are often inadequate, as pathogens develop resistance to various therapies and become particularly difficult to treat.

Pulmonary colonizations and infections typically become more prevalent and chronic as a patient suffering from CF ages (respiratory cultures from more than 80% of adults suffering from CF were positive for the presence of a pathogen). Therefore, the present method includes administering a polymer of the present invention before colonization or before an infection is acquired to prevent or inhibit onset of an infection. The method also includes treating a CF patient who is suffering from an active infection.

Colonizations and infections associated with CF are typically caused by a large variety of pathogens including Gram negative bacteria, Gram positive bacteria, fungi and viruses capable of infecting respiratory tract tissues. Bacteria and fungi associated with CF include, but are not limited to, Pseudomonas, Staphylococcus, Haemophilus, Burkholderia, Aspergillus, Candida, Mycobacteria, Mycoplasma, Stenotrophomonas, Escherichia, Achromobacter, Ralstonia, Acinetobacter, Streptococcus, Flavobacterium, Alcaligenes, CDC group VB3 and Klebsiella. Specific microbial species causing the colonization or infection include Pseudomonas aeruginosa, Staphylococcus aureus, Haemophilus influenzae, Burkholderia cepacia, Aspergillus fumigatus, Candida albicans, Mycoplasma pneumoniae, Stenotrophomonas maltophilia, Escherichia coli, Ralstonia mannitolilytica, Ralstonia pickettii, Streptococus pneumoniae, Flavobacterium indologenes, Burkholderia gladioli, Acinetobacter baumannii, Acinetobacter haemolyticus, Achromobacter xylosoxidans and Klebsiella pneumoniae. Viruses associated with CF include influenza virus (e.g., influenza virus A, influenza virus B, influenza virus C), respiratory syncytical virus and Rhinovirus. Pseudomonas aeruginosa is preferably treated or inhibited by the present method.

The polymer can be administered alone or in a pharmaceutical composition comprising the polymer, a pharmaceutically acceptable carrier, and optionally, one or more additional drugs, e.g., antibiotics or antimicrobials. Examples of co-therapies for infections or complications due to CF include tobramycin and other aminoglycosides, ciprofloacin and other fluoroquinolones, rifabutin, ethambutol, clarithromycin, clofazimine, aztreonam, cephalothin, cefazolin, nafcillin, ticarcilin, clavulanate, gentamicin, amikacin, ceftazidime, piperacillin, imipenem, cefepime, chloramphenicol, colistin, dicloxacillin, cefaclor, amoxicillin, azithromycin, trmethoprim/sulfa, cefpodoxime, tetracyclines, amiloride and meropenem. These antibiotics can be administered orally, by injection or by pulmonary means. The term “pulmonary” as used herein refers to any part, tissue or organ whose primary function is gas exchange with the external environment, i.e., O₂/CO₂ exchange, within a patient. “Pulmonary” typically refers to the tissues of the respiratory tract. Thus, the phrase “pulmonary administration” refers to administering the formulations described herein to any part, tissue or organ whose primary function is gas exchange with the external environment (e.g., mouth, nose, pharynx, oropharynx, laryngopharynx, larynx, trachea, carina, bronchi, bronchioles, alveoli). For purposes of the present invention, “pulmonary” is also meant to include a tissue or cavity that is contingent to the respiratory tract, in particular, the sinuses.

The polymer can also be administered with an anti-inflammatory drug or steroid such as ibuprofen, prednisone (corticosteroid) or pentoxifylline. Another suitable co-therapy is administering domase alfa (DNase), nacystelyn, gelsolin and hypertonic saline, which reduce mucus buildup, or administering a decongestant or bronchodilator (e.g, a beta adrenergic receptor agonist, an anticholinergic drug, theophylline).

The polymers of the present invention can also be administered in CF therapy following a physical therapy that aids mucus drainage. Such treatments include chest physiotherapy (manual or mechanical). Manual techniques include autogenic drainage and percussive techniques. Devices for mechanical therapy include positive expiratory pressure treatment, the “Flutter” mucus clearance device (a device that produces oscillations during exhalation), and an inflatable vest driven by a pulsed-air delivery system.

Polymers of the present invention can be administering to a patient suffering from CF in a manner as described above, but are preferably administered by pulmonary means (e.g., aerosol), intranasally or orally.

Conventional means to deliver the active agent by pulmonary means to a patient include administration of an aerosol formulation containing the active agent from, for example, a manual pump spray, nebulizer or pressurized metered-dose inhaler.

Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al., and WO 98/43650 by Montgomery, the entire teachings of which are incorporated herein by reference). Polymer compositions of the invention to be delivered as aerosols for treatment of pulmonary infection are formulated such that an effective dose may be aerosolized (e.g., using a jet or ultrasonic nebulizer) to a particle size optimal for treatment of pulmonary infections. Examples of a suitable particle size for delivery into the endobronchial space is generally about 1 to 5 microns.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

When administering the drug, the patient must actuate the drug delivery device. The actuation releases a fraction of the formulation from within the canister to the external environment. A force, created by vaporized propellant, expels the drug into the air and away from the device. The patient then inhales the aerosolized drug. The metering valve controls the amount of the formulation released, which, in turn, effectively controls the amount of drug available for inhalation by the patient.

Particles can also be administered by pulmonary means. To ensure that the drug particles have the proper size and shape, the particles may be analyzed using known techniques for determining particle morphology. For example, the particles can be visually inspected under a microscope and/or passed through a mesh screen. Preferred techniques for visualization of particles include scanning electron microscopy (SEM) and transmission electron microscopy (TEM). Particle size analysis may take place using laser diffraction methods. Commercially available systems for carrying out particle size analysis by laser diffraction are available from Clausthal-Zellerfeld, Germany (HELOS H1006).

Particles for pulmonary administration are typically substantially nonacicular particles. The particles will preferably have an average particle size in the range of about 0.5 micrometer to about 10 micrometer, more preferably in the range of about 1 micrometer to about 7.5 micrometer, and most preferably in the range of about 1 micrometer to about 5 micrometer. Preferably, greater than about 85%, more preferably greater than about 95%, and most preferably greater than about 98% of the population of particles in the formulation will fall within the desired particle size range, e.g., about 0.5 micrometer to about 10 micrometer, about 1 micrometer to about 7.5 micrometer, and so on.

Preferred drug delivery devices for particles are metered-dose inhalers. Metered-dose inhalers are described in Remington: The Science and Practice of Pharmacy, Twentieth Edition (Easton, Pa.: Mack Publishing Co., 2000) and in Ansel et al., Pharmaceutical Dosage Forms and Drug Delivery Systems, Sixth Edition (Malvern, Pa.: Lea & Febiger, 1995). The components of the drug delivery device, e.g., canister, housing, metering valve, etc., are commercially available. For example many components are available from 3M Corporation, St. Paul, Minn. Typically, although not necessarily, the amount of pharmaceutical formulation (including polymer, solvents and other expicipients) that is released per actuation of the drug delivery device is about 5 micrograms to about 100,000 micrograms of formulation.

The polymers are formulated in a manner appropriate for the route of administration. Typical formulations for pharmaceutical compositions are described above.

A polymer administered in the treatment or prevention of infection in CF patients generally has a weight between 500 and 5000 Daltons, 500 and 3000 Daltons, or 1000 and 3000 Daltons.

The ionene polymers and oligomers of the invention are also particularly useful for inhibiting the colonization or the growth and dissemination, of viruses, parasites and microorganisms, particularly on surfaces wherein such growth is undesirable. The term “inhibiting the growth of microorganisms” means that the growth, dissemination, accumulation, and/or the attachment, e.g. to a susceptible surface, of one or more viruses or microorganisms is impaired, retarded, eliminated or prevented. In a preferred embodiment, the antipathogenic compositions of the invention are used in methods for inhibiting the growth of an organism on susceptible surfaces in health-related environments. The term “health-related environment” as used herein includes all those environments where activities are carried out directly or indirectly, that are implicated in the restoration or maintenance of human health. A health-related environment can be a medical environment, where activities are carried out to restore human health. An operating room, a doctor's office, a hospital room, and a factory making medical equipment are all examples of health-related environments. Other health-related environments can include industrial or residential sites where activities pertaining to human health are carried out such as activities including food processing, water purification, recreational water maintenance, and sanitation.

The term “susceptible surface” as used herein refers to any surface whether in an industrial or medical setting, that provides an interface between an object and the fluid. A surface, as understood herein further provides a plane whose mechanical structure, without further treatment, is compatible with the adherence of microorganisms. Microbial growth and/or biofilm formation with health implications can involve those surfaces in all health-related environments. Such surfaces include, but are not limited to, scalpels, needles, scissors and other devices used in invasive surgical, therapeutic or diagnostic procedures; implantable medical devices, including artificial blood vessels, catheters and other devices for the removal or delivery of fluids to patients, artificial hearts, artificial kidneys, orthopedic pins, plates and implants; catheters and other tubes (including urological and biliary tubes, endotracheal tubes, peripherally insertable central venous catheters, dialysis catheters, long term tunneled central venous catheters, peripheral venous catheters, pulmonary catheters, Swan-Ganz catheters, urinary catheters, peritoneal catheters), urinary devices (including long term urinary devices, tissue bonding urinary devices, artificial urinary sphincters, urinary dilators), shunts (including ventricular or arterio-venous shunts); prostheses (including breast implants, penile prostheses, vascular grafting prostheses, heart valves, artificial joints, artificial larynxes, otological implants), vascular catheter ports, wound drain tubes, hydrocephalus shunts, pacemakers and implantable defibrillators, and the like.

Other surfaces include the inner and outer surfaces of pieces of medical equipment, medical gear worn or carried by personnel in the health care settings and protective clothing for biohazard or biological warfare applications. Such surfaces can include counter tops and fixtures in areas used for medical procedures or for preparing medical apparatus, tubes and canisters used in respiratory treatments, including the administration of oxygen, solubilized drugs in nebulizers, and anesthetic agents. Additional surfaces include those surfaces intended as biological barriers to infectious organisms such as gloves, aprons and faceshields.

Surfaces in contact with liquids are particularly prone to microbial growth or colonization and/or biofilm formation. As an example, those reservoirs and tubes used for delivering humidified oxygen to patients can bear biofilms inhabited by infectious agents. Dental unit waterlines similarly can bear biofilms on their surfaces, providing a reservoir for continuing contamination of the system of flowing and aerosolized water used in dentistry.

Other surfaces related to health include the inner and outer surfaces of equipment used in water purification, water storage and water delivery, and those articles involved in food processing equipment for home use, materials for infant care and toilet bowls.

In accordance with the invention, a method for preventing, inhibiting or eliminating the growth, dissemination, presence and/or accumulation of microorganisms on a susceptible surface (including but not limited to the formation of biofilms) comprises the step of contacting such surface with an antimicrobial/antipathogenic agent, or composition thereof of the invention, with an amount sufficient to prevent, inhibit or eliminate such growth, dissemination, presence and/or accumulation, i.e., with an effective amount.

As used herein “contacting” refers to any means for providing the compounds of the invention to a surface to be protected from, microbial growth and/or biofilm formation. Contacting can include spraying, wetting, immersing, dipping, painting, bonding, coating, adhering or otherwise providing a surface with a compound or composition in accordance with the invention. A “coating” refers to any temporary, semipermanent, or permanent layer, covering a surface. A coating can be a gas, vapor, liquid, paste, semisolid or solid. In addition a coating can be applied as a liquid and solidify into a hard coating. Examples of coatings include polishes, surface cleaners, caulks, adhesives, finishes, paints, waxes, polymerizable compositions (including phenolic resins, silicone polymers, chlorinated rubbers, coal tar and epoxy combinations, epoxy resins, polyamide resins vinyl resins, elastomers, acrylate polymers, fluoropolymers, polyesters and polyurethane, and latex). Silicone resins, silicone polymers (e.g. RTV polymers) and silicone heat cured rubbers are suitable coatings for use in the invention and described in the art. Coatings can be ablative or dissolvable, so that the dissolution rate of the matrix controls the rate at which the compositions of the invention are delivered to the surface. Coatings can also be non-ablative, and rely on diffusion principals to deliver a composition of the invention to the target surface. Non-ablative coatings can be porous or non-porous. A coating containing an agent of the invention freely dispersed in a polymer binder is referred to as a “monolithic” coating. Elasticity can be engineered into coatings to accommodate pliability, e.g. swelling or shrinkage of the surface to be coated.

Other means for contacting include a sustained or controlled release system that provides constant or prolonged release of an agent of the invention from a susceptible surface. This can be accomplished through the use of diffusional systems, including reservoir devices in which a core of an agent of the invention is surrounded by a porous membrane or layer, and also matrix devices in which the compound is distributed throughout an inert matrix. Materials which may be used to form reservoirs or matrices include silicones, acrylates, methacrylates, vinyl compounds such as polyvinyl chloride, olefins such as polyethylene or polypropylene, fluoropolymers such as polytetrafluorethylene or polypropylene, fluoropolymers such as polytetrafluorethylene, and polyesters such as terephthalates. Alternatively, the compositions of the invention may be mixed with a resin, e.g., polyvinyl chloride and then molded into a formed article, which integrally incorporates the compound to form a structure having a porous matrix which allows diffusion of the compound or a functional portion thereof into the surrounding environment. Microencapsulation techniques can also be used to maintain a sustained focal release of a compound of the invention.

Other means for providing the polyionene agents of the invention to a susceptible surface will be apparent to those of skill in the art.

The compounds and compositions of the invention are also useful for preventing microbial growth and/or biofilms in industries outside of health-related industries, such as industrial systems wherein the presence of an aqueous environment leads to biofilm formation. Examples of such systems include metal working fluids, cooling waters (e.g. intake cooling water, effluent cooling water, recirculating cooling water), and other recirculating water systems such as those used in papermaking or textile manufacture. Marine industries are also plagued by unwanted biofilms such as those that form on boat hulls and other marine structures. Another embodiment of the present invention is an article comprising a polymer of the present invention in an amount sufficient to prevent, inhibit or eliminate the growth or dissemination of a microorganism or the formation of a biofilm, i.e., an “effective amount.” The polymer can be in the article or on the surface of the article. Preferably, the article is coated with a composition comprising an effective amount of a polymer of the present invention. Articles that are advantageously coated with a polymer of the present invention are those in which inhibition of the growth of microorganisms and/or biofilms is desirable, e.g., medical devices, medical furniture and devices exposed to aqueous environments. Examples of such articles are described above.

One method of preparing a hydrolyzable polyionene involves reacting a diamine (e.g., an α,ω-diamine) with a diacrylate or other molecule having a carbonyl group alpha to a double bond. Examples of preparing a hydrolyzable polyionene by this method are found in Example 1.

Hydrolyzable ionene homopolymers of the present invention can also be prepared by reacting a divalent electrophile, where the electrophile contains one or more hydrolyzable groups, with a diamine or a diphosphine. Preferably, the diamine or diphosphine is secondary or tertiary.

Homopolymers of the present invention consist of one ionene monomer and one hydrolyzable monomer. Copolymers can be prepared by reacting two or more ionene monomers and/or two or more hydrolyzable monomers. Alternatively, a copolymer can contain a monomeric unit that is not an ionene and is not hydrolyzable, in addition to the required ionene and hydrolyzable monomers.

“Capping groups” are typically present at the end of a polyionene, which result from a partially reacted divalent electrophile or nucleophile or a monovalent electrophile or nucleophile. Capping groups can optionally be reacted further. For example, capping groups having a primary, secondary or tertiary heteroatom (e.g., N, P) can be reacted with an alkylating agent or an acylating agent or can be reacted with an acid listed above to form a salt.

Ionene polymers of the invention can be cross-linked with primary, secondary or other polyfunctional amines using means known in the art. Ionene polymers can be cross-linked by polymerizing in the presence of a multivalent nucleophile (i.e., a compound with three or more nucleophilic groups such as a triamine or tetraamine) or a multivalent electrophile (i.e., a compound with three or more nucleophilic groups such as a trihalide or tetrahalide).

### EXEMPLIFICATION

### Example 1

Procedure for Synthesizing an Array of 96 Hydrolyzable Polyionenes

Stock solutions (3.2M) of eight different secondary diamines and 12 different diacrylates, all of which are shown below, were prepared in methylene chloride. One ml of each sample was added to 8 ml tared and labeled vials which were set up in an 8×12 array. Materials that would not dissolve in methylene chloride were added neat to their appropriate vials (3.2 mmol), as well as 1 ml of methylene chloride. The samples were placed on a heater/shaker block at a temperature of 45° C. and shaken for 5 days. During this reaction period, 0.5 ml of methylene chloride was added to any sample that was not completely dissolved. After the five days, the samples were cooled to room temperature and precipitated with ether. Approximately 3 ml of ether were added to each sample. The sample was vortexed and allowed to settle. The ether was decanted off and methylene chloride was added to dissolve each sample again. This precipitation procedure was repeated a total of 5 times. If the sample dissolved in ether, hexane was used for the precipitation step. The samples were dried on the speed vacuum at 45° C. overnight. A yield was obtained. A small amount of the sample (˜10 mg) was placed in a scintillation vial. Two and one-half ml of 25 mM acetate buffer was added to each to determine solubility.

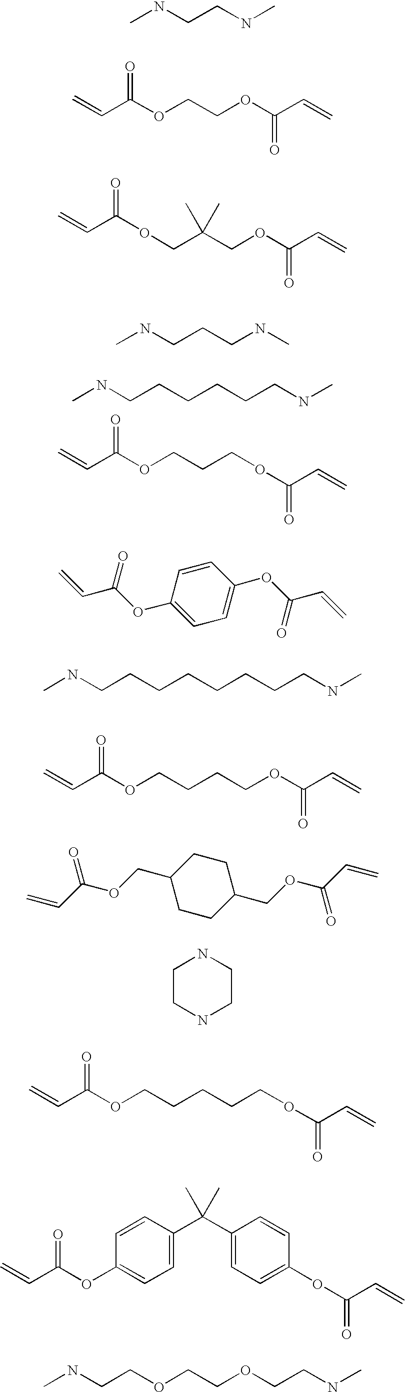

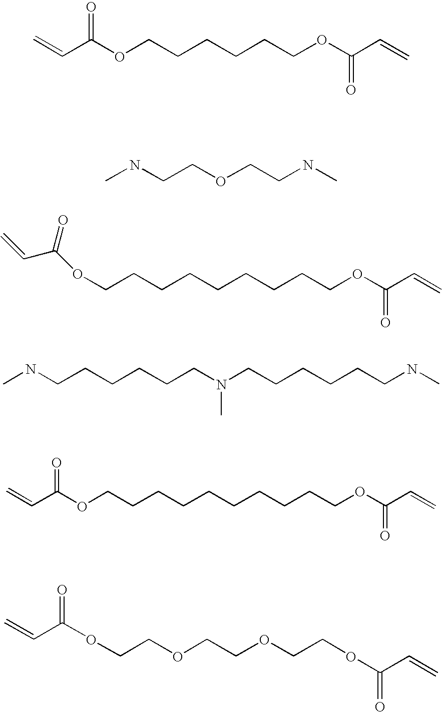

While this invention has been particularly shown and described with references to preferred embodiments thereof, it will be understood by those skilled in the art that various changes in form and details may be made therein without departing from the scope of the invention encompassed by the appended claims.

What is claimed is:

1. An ionene polymer comprising hydrolyzable groups in the polymer backbone, wherein said ionene polymer contains tertiary or quaternary phosphorus atoms or quaternary nitrogen atoms, and wherein each hydrolysable group is independently is an ester, a carbonate, a carbamate, an orthoester, an orthocarbonate, an acetal, a ketal, a phosphazene, a carboxyacetal, a carboxyorthoester, a thioorthoester, a sulfoxyorthoester or an alpha-hydroxy acid.

2. A hydrolyzable ionene polymer comprised of repeat units represented by Structural Formula (I) and (II): 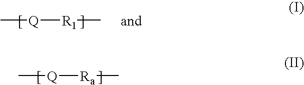
wherein:
each R₁ is a linker;
each Rₐ is independently a hydrolyzable group or a substituted or unsubstituted hydrocarbyl group interrupted with one or more hydrolyzable groups;
each Q is independently: 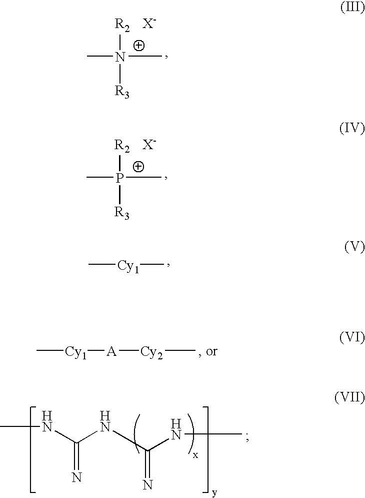
Cy₁ and Cy₂ are each independently a quaternary nitrogen-containing monocyclic heteroaromatic ring, a tertiary nitrogen-containing non-aromatic ring or a quaternary nitrogen-containing non-aromatic heterocyclic ring;
A is a covalent bond, or a substituted or unsubstituted lower alkylene group;
R₂ and R₃ are independently a substituted or unsubstituted aliphatic or aromatic group;
each X⁻, separately or taken together with other X⁻s, is a physiologically acceptable anion;
x is an integer from 0-4; and
y is an integer from 1-5.

3. The polymer of claim 2, wherein Q of Structural Formula (I) is the same as Q of Structural Formula (II).

4. The polymer of claim 3, wherein Rₐ is a hydrolyzable group or a substituted or unsubstituted alkylene group interrupted with one or more hydrolyzable groups.

5. The polymer of claim 4, wherein the hydrolyzable group is an ester, a carbonate, a carbamate, an orthoester, an orthocarbonate, an acetal, a ketal, a phosphazene, a carboxyacetal, a carboxyorthoester, a thioorthoester, a sulfoxyorthoester or an alpha-hydroxy acid.

6. The polymer of claim 4, wherein the polymer has a molecular weight from 500 Daltons to 20,000 Daltons.

7. The polymer of claim 6, wherein the polymer has a molecular weight from 1,000 Daltons to 5,000 Daltons.

8. The polymer of claim 7, wherein the polymer has a molecular weight from 1,000 Daltons to 3,000 Daltons.

9. The polymer of claim 4, wherein each R₂ and R₃ are each independently an alkyl group or a hydroxyalkyl group.

10. The polymer of claim 9, wherein Q is represented by Structural Formula (III):

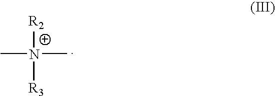

11. The polymer of claim 10, wherein R₁ is a substituted or unsubstituted straight chained lower alkylene group or polyalkylene glycol, wherein the lower alkyl group or the polyalkylene glycol is optionally substituted with one or more —OH groups.

12. The polymer of claim 4, wherein Q is represented by the formula: 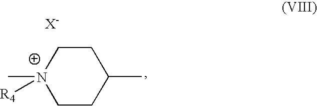
wherein R₄ is an electron pair, hydrogen or a substituted or unsubstituted lower alkyl group.

13. The polymer of claim 12, wherein R₄ is a lower alkyl or hydroxy substituted lower alkyl group.

14. The polymer of claim 4, wherein Q is represented by the formula: 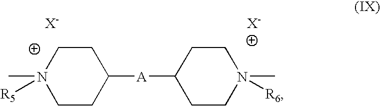
wherein A is a bond or substituted or unsubstituted lower alkylene group, and wherein R₅ and R₆ are each independently an electron pair, hydrogen or a substituted or unsubstituted lower alkyl group.

15. The polymer of claim 14, wherein R₅ and R₆ are each independently an alkyl group or a hydroxyalkyl group.

16. The polymer of claim 15, wherein A is an unsubstituted straight chained lower alkylene group.

17. The polymer of claim 16, wherein R₁ is a substituted or unsubstituted straight chained lower alkylene group or polyalkylene glycol optionally substituted with one or more —OH groups.

18. The polymer of claim 17, wherein the repeat unit represented by Structural Formula (I) has the formula:

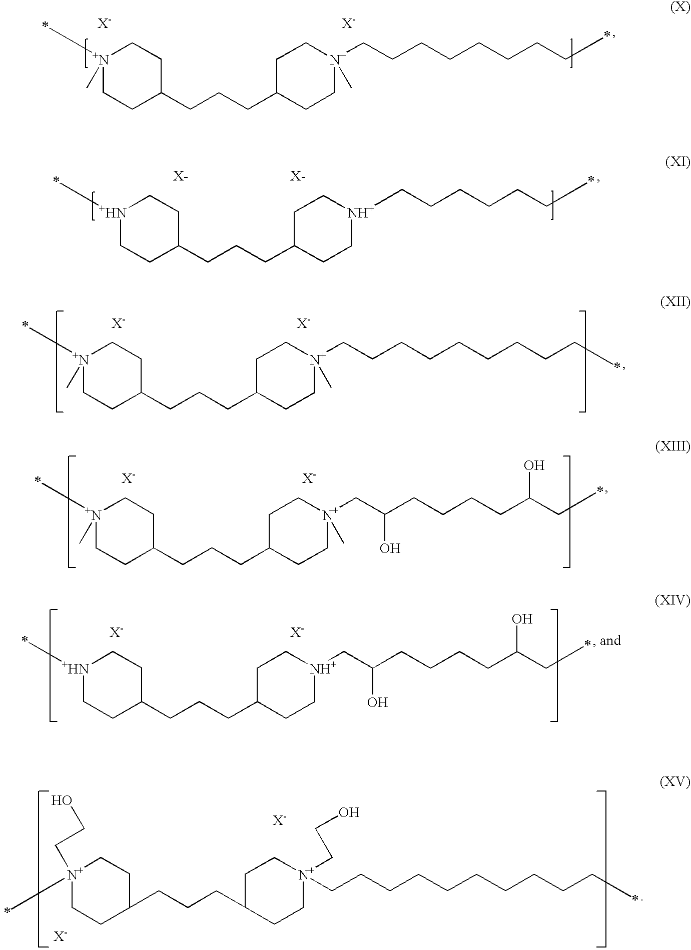

19. The polymer of claim 17, wherein R₁ is an unsubstituted polyalkylene glycol or —CH₂CHOH(CH₂)ₙCHOHCH₂— wherein n is an integer from 0 to 8.

20. The polymer of claim 4, wherein Q is represented by Structural Formula (I): 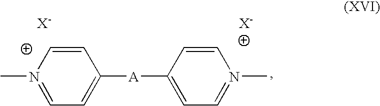
wherein A is a bond or a substituted or unsubstituted lower alkylene or lower alkylene glycol group.

21. The polymer of claim 20, wherein A is an unsubstituted straight chained lower alkylene group.

22. The polymer of claim 21, wherein R₁ is a substituted or unsubstituted straight chained lower alkylene group or polyalkylene glycol optionally substituted with one or more —OH groups.

23. The polymer of claim 22, wherein the repeat unit represented by Structural Formula (I) has the formula:

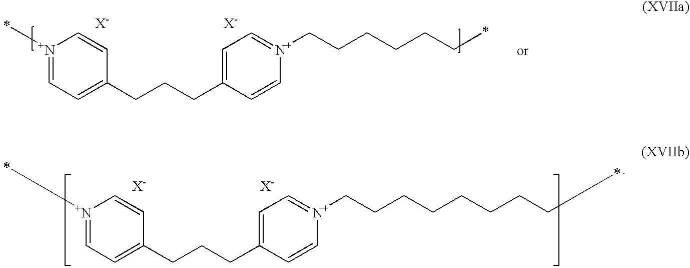

24. The polymer of claim 22, wherein R₁ is an unsubstituted polyalkylene glycol or —CH₂CHOH(CH₂)ₙCHOHCH₂— wherein n is an integer from 0 to 8.

25. The polymer of claim 24, wherein the repeat unit represented by Structural Formula (I) has the formula:

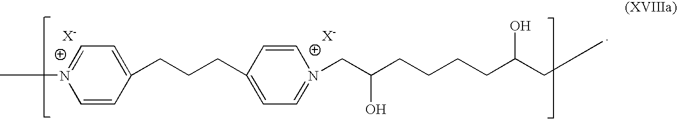

26. The polymer of claim 4, wherein repeat units represented by Structural Formula (I) have the formula: 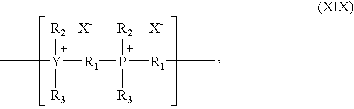
wherein:
Y is P or N;
R₁ is a substituted or unsubstituted hydrocarbyl group;
R₂ and R₃ are independently a substituted or unsubstituted aliphatic or aromatic group;
each X⁻ in the polymer or copolymer, separately or taken together with other X⁻s, is a physiologically acceptable anion.

27. The polymer of claim 26, wherein the repeat units represented by Structural Formula (I) have the formula: 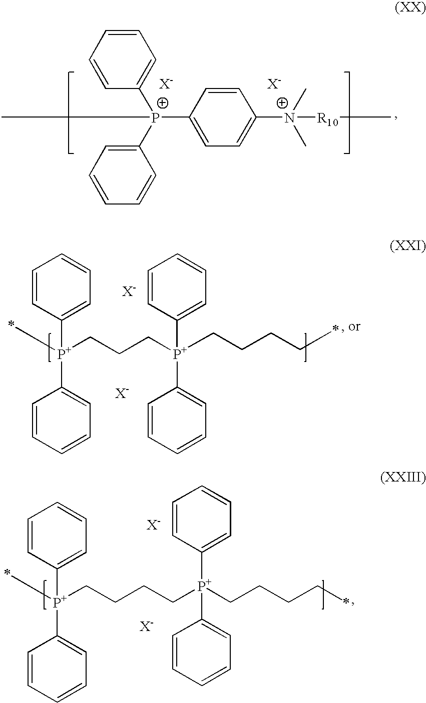
wherein R₁₀ is a substituted or unsubstituted lower alkylene group having from 4 to 12 carbon atoms and each X⁻, separately or taken together with other X⁻s is a physiologically acceptable anion.

28. The polymer of claim 4, wherein Q is represented by Structural Formula (VII):

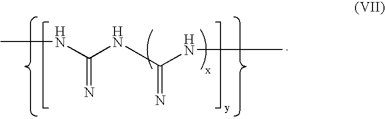

29. The polymer of claim 28, wherein the repeat units represented by Structural Formula (I) have the formula:

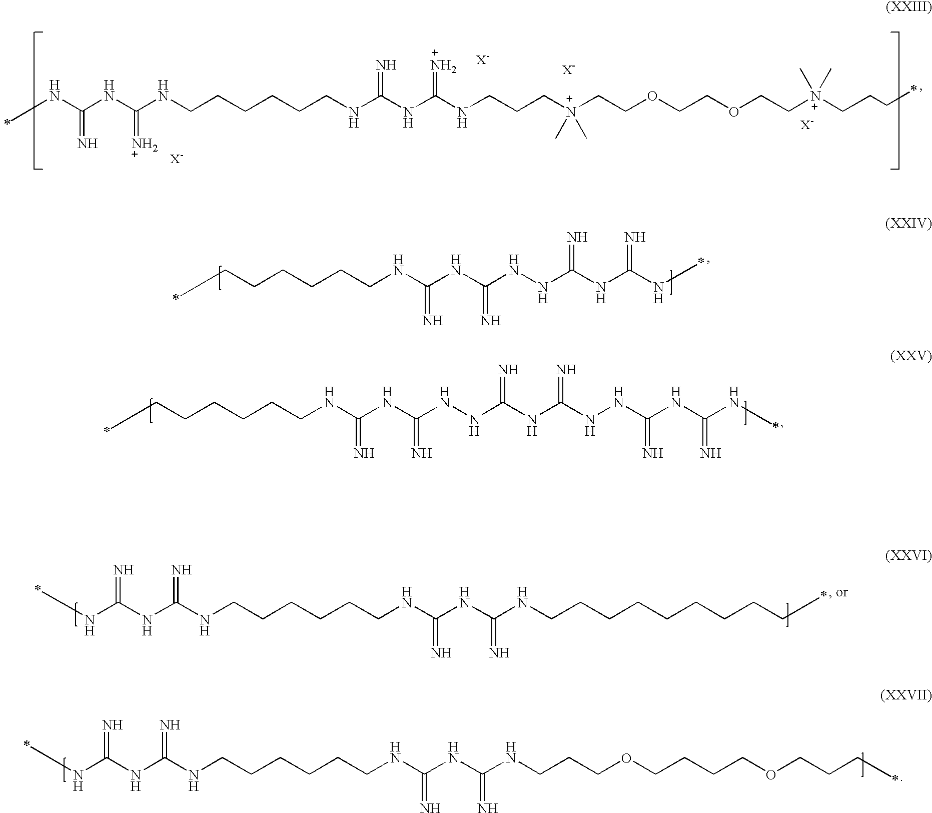

30. The polymer of claim 5, wherein each hydrolyzable group is an ester, carbonate or carbamate.

31. The polymer of claim 5, wherein the repeat unit represented by Structural Formula (II) is represented by the structural formula:

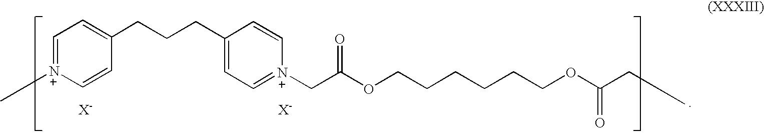

32. The hydrolyzable ionene polymer of claim 5, where the repeat units represented by Structural Formula (II) are represented by the structural formula: 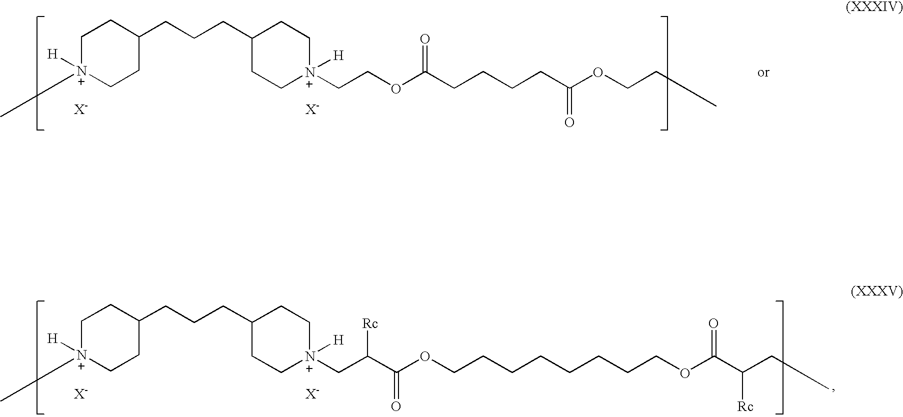
wherein R_{c} is a substituted or unsubstituted alkyl group.

33. The polymer of claim 4, wherein the polymer consists of repeat units represented by Structural Formula (I) and Structural Formula (II).

34. The polymer of claim 33, wherein the polymer consists of a 1:1 ratio of repeat units represented by Structural Formula (I) and Structural Formula (II).

35. The polymer of claim 4, wherein the polymer comprises 10-90 mole % repeat units represented by Structural Formula (I) and 10-90 mole % repeat units represented by Structural Formula (II).

36. The polymer of claim 35, wherein the polymer comprises 10-70 mole % repeat units represented by Structural Formula (I) and 30-90 mole % repeat units represented by Structural Formula (II).

37. The polymer of claim 36, wherein the polymer comprises 10-50 mole % repeat units represented by Structural Formula (I) and 50-90 mole % repeat units represented by Structural Formula (II).

38. A hydrolyzable ionene homopolymer comprised of repeat units represented by Structural Formula (II): 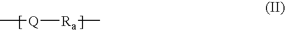
wherein:
each Rₐ is independently a hydrolyzable group or a substituted or unsubstituted hydrocarbyl group interrupted with one or more hydrolyzable groups;
each hydrolysable group is independently an ester, a carbonate, a carbamate, an orthoester, an orthocarbonate, an acetal, a ketal, a phosphazene, a carboxyacetal, a carboxyorthoester, a thioorthoester, a sulfoxyorthoester or an alpha-hydroxy acid;
each Q is independently: 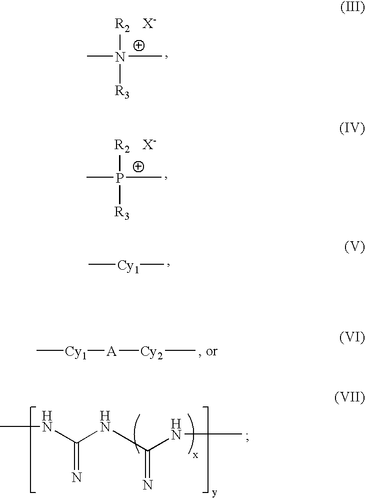
Cy₁ and Cy₂ are each independently a quaternary nitrogen-containing monocyclic heteroaromatic ring or a quaternary nitrogen-containing non-aromatic heterocyclic ring;
A is a covalent bond, or a substituted or unsubstituted lower alkylene group;
R₂ and R₃ are independently a substituted or unsubstituted aliphatic or aromatic group;
each X⁻, separately or taken together with other X⁻s, is a physiologically acceptable anion;
x is an integer from 0-4; and
y is an integer from 1-5.

39. The polymer of claim 38, wherein the hydrolyzable group is an ester, a carbonate or a carbamate.

40. The polymer of claim 39, wherein each hydrolyzable group is an ester.

41. The polymer of claim 39, wherein each hydrolyzable group is a carbonate.

42. The polymer of claim 39, wherein each hydrolyzable group is a carbamate.

43. The polymer of claim 39, wherein the polymer has a molecular weight from 500 Daltons to 20,000 Daltons.

44. The polymer of claim 40, wherein the repeat unit is represented by Structural Formula (XXVIII) or (XXIX): 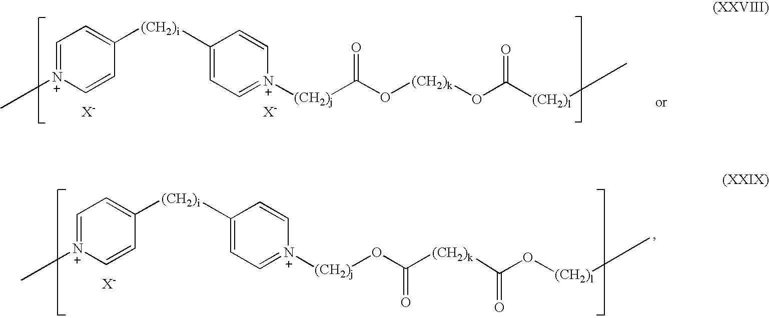
wherein i and j are each independently an integer ranging from 0 to 8 and k and l are each independently an integer ranging from 1 to 8.

45. The polymer of claim 44, wherein k is an integer from 1 to 3.

46. The polymer of claim 40, wherein the repeat unit is represented by Stuctural Formula (XXX) or (XXXI): 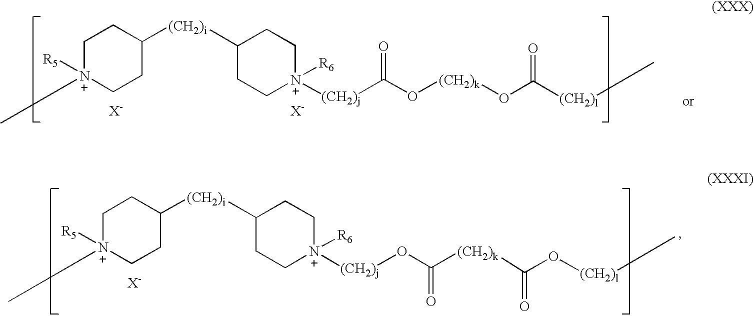
wherein R₅ and R₆ are each independently a substituted or unsubstituted lower alkyl group and i and j are each independently an integer ranging from 0 to 8 and k and l are each independently an integer ranging from 1 to 8.

47. The polymer of claim 46, wherein R₅ and R₆ are the same and k is an integer ranging from 1 to 3.

48. A pharmaceutical composition comprising a carrier or diluent and an ionene polymer comprising hydrolyzable groups in the polymer backbone, wherein said ionene polymer contains tertiary or quaternary phosphorus atoms or quaternary nitrogen atoms, and wherein each hydrolysable group is independently an ester, a carbonate, a carbamate, an orthoester, an orthocarbonate, an acetal, a ketal, a phosphazene, a carboxyacetal, a carboxyorthoester, a thioorthoester, a sulfoxyorthoester or an alpha-hydroxy acid.

49. A pharmaceutical composition comprising a carrier or diluent and a polymer comprised of repeat units represented by Structural Formula (I) and (II): 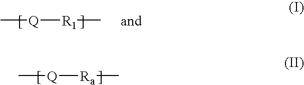
wherein:
each R₁ is independently a linker;
each Rₐ is independently a hydrolyzable group or a substituted or unsubstituted hydrocarbyl group interrupted with one or more hydrolyzable groups;
each Q is independently: 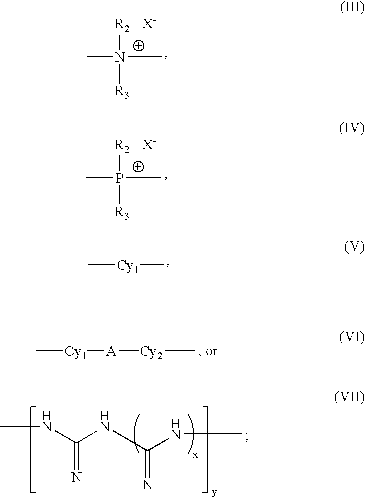
Cy₁ and Cy₂ are each independently a quaternary nitrogen-containing monocyclic heteroaromatic ring, a tertiary nitrogen-containing non-aromatic ring or a quaternary nitrogen-containing non-aromatic heterocyclic ring;
A is a covalent bond, or a substituted or unsubstituted lower alkylene group;
R₂ and R₃ are independently a substituted or unsubstituted aliphatic or aromatic group;
each X⁻, separately or taken together with other X⁻s, is a physiologically acceptable anion;
x is an integer from 0-4; and
y is an integer from 1-5.

50. The pharmaceutical composition of claim 49, wherein Q of Structural Formula (I) is the same as Q of Structural Formula (II).

51. The pharmaceutical composition of claim 50, wherein Rₐ is a hydrolyzable group or a substituted or unsubstituted alkylene group interrupted with one or more hydrolyzable groups.

52. The pharmaceutical composition of claim 51, wherein the hydrolyzable group is an ester, a carbonate or a carbamate.

53. The pharmaceutical composition of claim 51, wherein each R₂ and R₃ are each independently an alkyl group or a hydroxyalkyl group.

54. The pharmaceutical composition of claim 53, wherein Q is represented by Structural Formula (III):

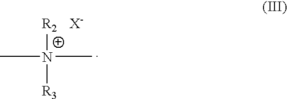

55. The pharmaceutical composition of claim 51, wherein Q is represented by the formula: 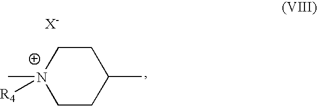
wherein R₄ is an electron pair, hydrogen or a substituted or unsubstituted lower alkyl group.

56. The pharmaceutical composition of claim 51, wherein Q is represented by the formula: 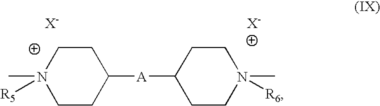
wherein A is a bond or substituted or unsubstituted lower alkylene group, and wherein R₅ and R₆ are each an electron pair, hydrogen or a substituted or unsubstituted lower alkyl group.

57. The pharmaceutical composition of claim 51, wherein Q is represented by the formula: 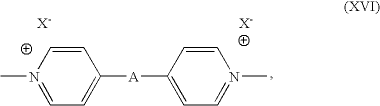
wherein A is a bond or a substituted or unsubstituted lower alkylene or lower alkylene glycol group.

58. The pharmaceutical composition of claim 51, wherein repeat units represented by Structural Formula (I) have the formula: 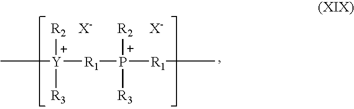
wherein:
Y is P or N;
R₁ is a substituted or unsubstituted hydrocarbyl group;
R₂ and R₃ are independently a substituted or unsubstituted aliphatic or aromatic group;
each X⁻ in the polymer or copolymer, separately or taken together with other X⁻s, is a physiologically acceptable anion.

59. The pharmaceutical composition of claim 38, wherein Q is represented by Structural Formula (VII):

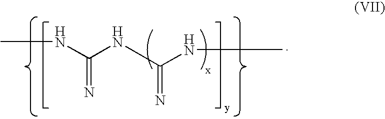

60. A pharmaceutical composition comprising a carrier or diluent and a polymer comprised of repeat units represented by Structural Formula (II): 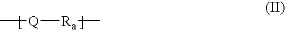
wherein:
each Rₐ is independently a hydrolyzable group or a substituted or unsubstituted hydrocarbyl group interrupted with one or more hydrolyzable groups;
each hydrolysable group is independently an ester, a carbonate, a carbamate, an orthoester, an orthocarbonate, an acetal, a ketal, a phosphazene, a carboxyacetal, a carboxyorthoester, a thioorthoester, a sulfoxyorthoester or an alpha-hydroxy acid;
each Q is independently: 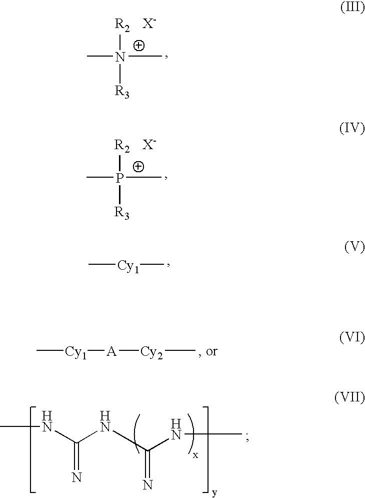
Cy₁ and Cy₂ are each independently a quaternary nitrogen-containing monocyclic heteroaromatic ring or a quaternary nitrogen-containing non-aromatic heterocyclic ring;
A is a covalent bond, or a substituted or unsubstituted lower alkylene group;
R₂ and R₃ are independently a substituted or unsubstituted aliphatic or aromatic group;
each X⁻, separately or taken together with other X⁻s, is a physiologically acceptable anion;
x is an integer from 0-4; and
y is an integer from 1-5.

61. The polymer of claim 60, wherein the hydrolyzable group is an ester, a carbonate or a carbamate.

62. A method of treating a viral, parasitic or microbial infection in a mammal comprising the step of administering to said mammal an effective amount of an ionene polymer comprising hydrolyzable groups in the polymer backbone.

63. A method of treating a viral, parasitic or microbial infection in a mammal comprising the step of administering to said mammal an effective amount of a polymer comprised of repeat units represented by Structural Formula (II) or Structural Formula (I) and (II): 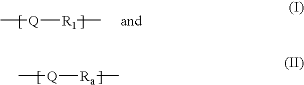
wherein:
each R₁ is independently a linker;
each Rₐ is independently a hydrolyzable group or a substituted or unsubstituted hydrocarbyl group interrupted with one or more hydrolyzable groups;
each Q is independently: 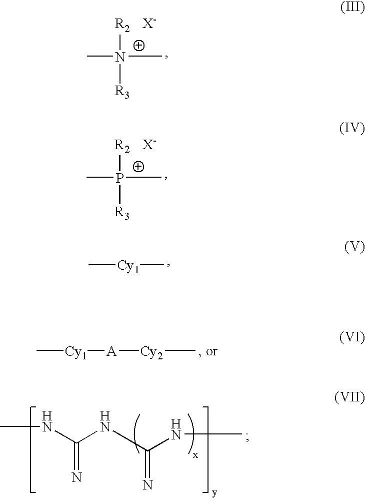
Cy₁ and Cy₂ are each independently a quaternary nitrogen-containing monocyclic heteroaromatic ring, a tertiary nitrogen-containing non-aromatic ring or a quaternary nitrogen-containing non-aromatic heterocyclic ring;
A is a covalent bond, or a substituted or unsubstituted lower alkylene group;
R₂ and R₃ are independently a substituted or unsubstituted aliphatic or aromatic group;
each X⁻, separately or taken together with other X⁻s, is a physiologically acceptable anion;
x is an integer from 0-4; and
y is an integer from 1-5.

64. The method of claim 63, wherein the microbial infection is a bacterial infection.

65. The method of claim 63, wherein the infection is a viral infection.

66. The method of claim 63, wherein the microbial infection is a protozoal infection.

67. The method of claim 63, wherein the microbial infection is a fungal infection.

68. The method of claim 63, wherein the polymer is administered orally, buccally, ophthalmically or topically.

69. The method of claim 63, wherein Rₐ is a hydrolyzable group or a substituted or unsubstituted alkylene group interrupted with one or more hydrolyzable groups.

70. The method of claim 69, wherein the hydrolyzable group is an ester, a carbonate or a carbamate.

71. The method of claim 69, wherein each R₂ and R₃ are each independently an alkyl group or a hydroxyalkyl group.

72. The method of claim 70, wherein Q is represented by Structural Formula (III):

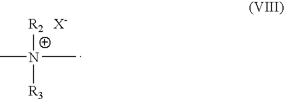

73. The method of claim 69, wherein Q is represented by the formula: 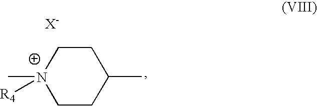
wherein R₄ is an electron pair, hydrogen or a substituted or unsubstituted lower alkyl group.

74. The method of claim 69, wherein Q is represented by the formula: 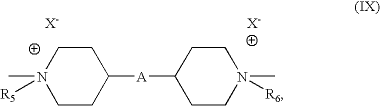
wherein A is a bond or substituted or unsubstituted lower alkylene group, and wherein R₅ and R₆ are each independently an electron pair, hydrogen or a substituted or unsubstituted lower alkyl group.

75. The method of claim 69, wherein Q is represented the formula: 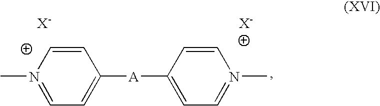
wherein A is a bond or a substituted or unsubstituted lower alkylene or lower alkylene glycol group.

76. The method of claim 69, wherein repeat units represented by Structural Formula (I) have the formula: 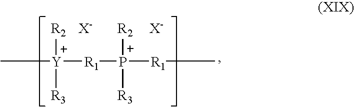
wherein:
Y is P or N;
R₁ is a substituted or unsubstituted hydrocarbyl group;
R₂ and R₃ are independently a substituted or unsubstituted aliphatic or aromatic group;
each X⁻ in the polymer or copolymer, separately or taken together with other X⁻s, is a physiologically acceptable anion.

77. The method of claim 69, wherein Q is represented by Structural Formula (VII):

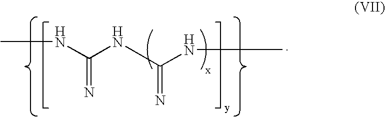

78. A method of inhibiting the growth of a virus, parasite or microorganism on a surface comprising the step of contacting said surface with an effective amount of an ionene polymer comprising hydrolyzable groups in the polymer backbone.

79. A method of inhibiting the growth of a virus, parasite or microorganism on a surface comprising the step of contacting said surface with an effective amount of a polymer comprised of repeat units represented by Structural Formula (II) or Structural Formula (I) and (II): 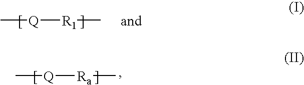
wherein:
each R₁ is independently a linker;
each Rₐ is independently a hydrolyzable group or a substituted or unsubstituted hydrocarbyl group interrupted with one or more hydrolyzable groups;
each Q is independently: 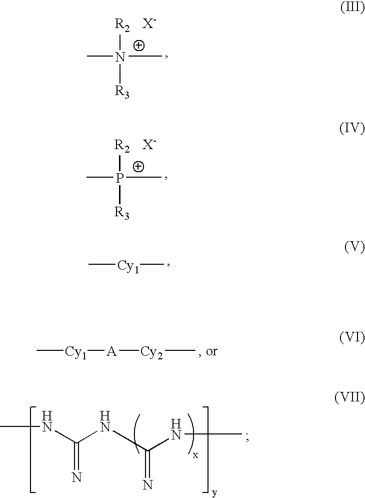
Cy₁ and Cy₂ are each independently a quaternary nitrogen-containing monocyclic heteroaromatic ring, a tertiary nitrogen-containing non-aromatic heterocyclic ring or a quaternary nitrogen-containing non-aromatic heterocyclic ring;
A is a covalent bond, or a substituted or unsubstituted lower alkylene group;
R₂ and R₃ are independently a substituted or unsubstituted aliphatic or aromatic group;
each X⁻, separately or taken together with other X⁻s, is a physiologically acceptable anion;
x is an integer from 0-4; and
y is an integer from 1-5.

80. The method of claim 79, wherein the surface is in contact with a liquid.

81. The method of claim 79, wherein the surface is in a health-related environment.

82. The method of claim 81, wherein the surface is that of a device used in invasive surgical, therapeutic or diagnostic procedures.

83. The method of claim 82, wherein the device is an implantable medical device.

84. The method of claim 79, wherein the surface is a biological barrier for an infectious organism.

85. The method of claim 79, wherein the microorganism is a bacterium.

86. The method of claim 85, wherein the bacterium is a Staphylococcus, Salmonella, Listeria or Bacillus species or Escherichia coli.

87. The method of claim 79, wherein the growth of a virus is inhibited.

88. The method of claim 79, wherein the microorganism is a protist.

89. The method of claim 79, wherein the microorganism is a fungus.

90. The method of claim 79, wherein Rₐ is a hydrolyzable group or a substituted or unsubstituted alkylene group interrupted by one or more hydrolyzable groups.

91. The method of claim 90, wherein the hydrolyzable group is an ester, a carbonate or a carbamate.

92. The method of claim 90, wherein each R₂ and R₃ are each independently an alkyl group or a hydroxyalkyl group.

93. The method of claim 92, wherein Q is represented by Structural Formula (III):

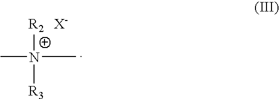

94. The method of claim 90, wherein Q is represented by the formula: 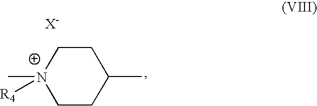
wherein R₄ is an electron pair, hydrogen or a substituted or unsubstituted lower alkyl group.

95. The method of claim 90, wherein Q is represented by the formula: 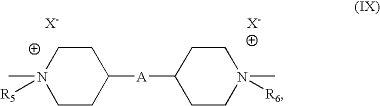
wherein A is a bond or substituted or unsubstituted lower alkylene group, and wherein R₅ and R₆ are each independently an electron pair, hydrogen or a substituted or unsubstituted lower alkyl group.

96. The method of claim 90, wherein Q is represented by the formula: 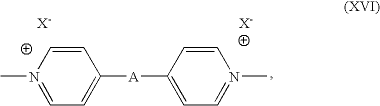
wherein A is a bond or a substituted or unsubstituted lower alkylene or lower alkylene glycol group.

97. The method of claim 90, wherein repeat units represented by Structural Formula (I) have the formula: 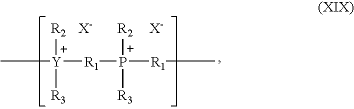
wherein:
Y is P or N;
R₁ is a substituted or unsubstituted hydrocarbyl group;
R₂ and R₃ are independently a substituted or unsubstituted aliphatic or aromatic group;
each X⁻ in the polymer or copolymer, separately or taken together with other X⁻s, is a physiologically acceptable anion.

98. The method of claim 90, wherein Q is represented by Structural Formula (VII):

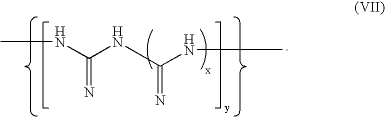

99. A method of treating mucositis in a mammal comprising the step of administering to said mammal an effective amount of an ionene polymer comprising hydrolyzable groups in the polymer backbone.

100. A method of treating mucositis in a mammal comprising the step of administering to said mammal an effective amount of a polymer comprised of repeat units represented by Structural Formula (II) or Structural Formula (I) and (II): 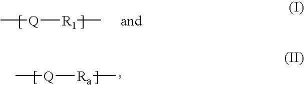
wherein:
each R₁ is independently a substituted or unsubstituted hydrocarbyl group optionally interrupted with one or more heteroatoms;
each Rₐ is independently a hydrolyzable group or a substituted or unsubstituted hydrocarbyl group optionally interrupted with one or more hydrolyzable groups;
each Q is independently: 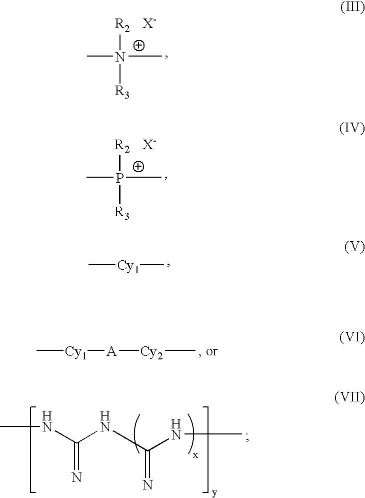
Cy₁ and Cy₂ are each independently a quaternary nitrogen-containing monocyclic heteroaromatic ring, a tertiary nitrogen-containing non-aromatic heterocyclic ring or a quaternary nitrogen-containing non-aromatic heterocyclic ring;
A is a covalent bond, or a substituted or unsubstituted lower alkylene group;
R₂ and R₃ are independently a substituted or unsubstituted aliphatic or aromatic group;
each X⁻, separately or taken together with other X⁻s, is a physiologically acceptable anion;
x is an integer from 0-4; and
y is an integer from 1-5.

101. The method of claim 100, wherein the polymer is administered therapeutically.

102. The method of claim 100, wherein the polymer is administered prophylactically.

103. The method of claim 100, wherein the polymer is administered orally, buccally, rectally, vaginally or intranasally.

104. The method of claim 100, wherein said mucositis is oral mucositis.

105. The method of claim 104, wherein said oral mucositis is a side effect of anti-cancer therapy.

106. The method of claim 105, wherein said anti-cancer therapy is chemotherapy or radiation therapy.

107. The method of claim 104, wherein said oral mucositis is a side effect of bone marrow transplantation or stem cell transplantation or ablation.

108. The method of claim 104, wherein Rₐ is a hydrolyzable group or a substituted or unsubstituted alkylene group interrupted with one or more hydrolyzable groups.

109. The method of claim 108, wherein the hydrolyzable group is an ester, a carbonate or a carbamate.

110. The method of claim 108, wherein each R₂ and R₃ are each independently an alkyl group or a hydroxyalkyl group.

111. The method of claim 110, wherein Q is represented by Structural Formula (III):

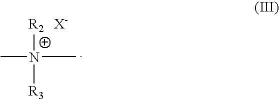

112. The method of claim 108, wherein Q is represented by the formula: 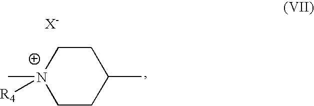
wherein R₄ is an electron pair, hydrogen or a substituted or unsubstituted lower alkyl group.

113. The method of claim 108, wherein Q is represented by the formula: 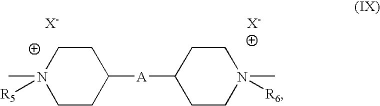
wherein A is a bond or substituted or unsubstituted lower alkylene group, and wherein R₅ and R₆ are each independently an electron pair, hydrogen or a substituted or unsubstituted lower alkyl group.

114. The method of claim 108, wherein Q is represented by the formula: 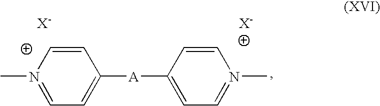
wherein A is a bond or a substituted or unsubstituted lower alkylene or lower alkylene glycol group.

115. The method of claim 114, wherein the repeat unit represented by Structural Formula (I) has the formula:

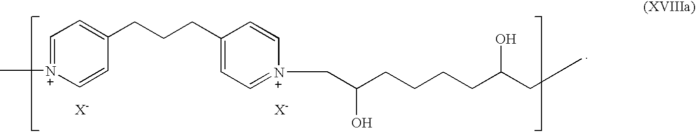

116. The method of claim 108, wherein repeat units represented by Structural Formula (I) have the formula: 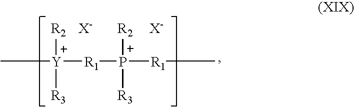
wherein:
Y is P or N;
R₁ is a substituted or unsubstituted hydrocarbyl group;
R₂ and R₃ are independently a substituted or unsubstituted aliphatic or aromatic group;
each X⁻ in the polymer or copolymer, separately or taken together with other X⁻s, is a physiologically acceptable anion.

117. The method of claim 108, wherein Q is represented by Structural Formula (VII):

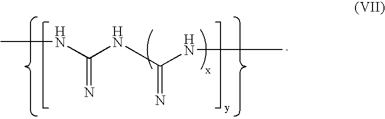

118. A method of preventing or treating infection or inhibiting or preventing colonization in a cystic fibrosis patient comprising the step of administering to said patient an effective amount of an ionene polymer comprising hydrolyzable groups in the polymer backbone.

119. A method of preventing or treating infection or colonization in a cystic fibrosis patient comprising the step of administering to said patient an effective amount of a polymer comprised of repeat units represented by Structural Formula (II) or Structural Formula (I) and (II): 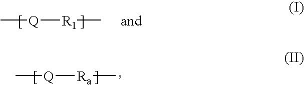
wherein:
each R₁ is independently a linker;
each Rₐ is independently a hydrolyzable group or a substituted or unsubstituted hydrocarbyl group interrupted with one or more hydrolyzable groups;
each Q is independently: 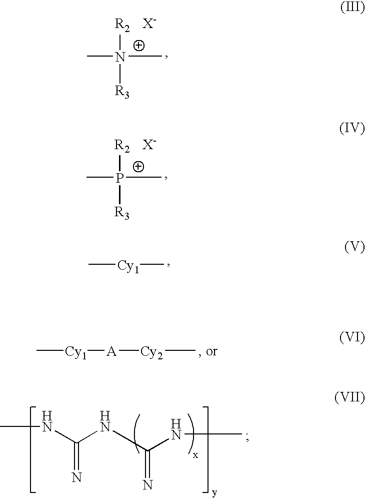
Cy₁ and Cy₂ are each independently a quaternary nitrogen-containing monocyclic heteroaromatic ring, a tertiary nitrogen-containing non-aromatic heterocyclic ring or a quaternary nitrogen-containing non-aromatic heterocyclic ring;
A is a covalent bond, or a substituted or unsubstituted lower alkylene group;
R₂ and R₃ are independently a substituted or unsubstituted aliphatic or aromatic group;
each X⁻, separately or taken together with other X⁻s, is a physiologically acceptable anion;
x is an integer from 0-4; and
y is an integer from 1-5.

120. The method of claim 119, wherein the patient is suffering from a pulmonary infection or colonization.

121. The method of claim 120, wherein the polymer is administered as an aerosol.

122. The method of claim 121, wherein the molecular weight of the polymer is 1000 to 3000 Daltons.

123. The method of claim 121, wherein the infection or colonization is caused by a microbe selected from the group consisting of Pseudomonas, Staphylococcus, Haemophilus, Burkholderia, Aspergillus, Candida, Mycobacteria, Mycoplasma, Stenotrophomonas, Escherichia, Achromobacter, Ralstonia, Acinetobacter, Streptococcus, Flavobacterium or Klebsiella species, and combinations thereof.

124. The method of claim 123, wherein the microbe is selected from the group consisting of Pseudomonas aeruginosa, Staphylococcus aureus, Haemophilus influenzae, Burkholderia cepacia, Aspergillus fumigatus, Candida albicans, Mycoplasma pneumoniae, Stenotrophomonas maltophilia, Escherichia coli, Klebsiella pneumoniae, Ralstonia mannitolilytica, Ralstonia pickettii, Streptococcus pneumoniae, Flavobacterium indologenes, Burkholderia gladioli, Acinetobacter baumannii, Achromobacter xylosoxidans, and combinations thereof.

125. The method of claim 121, wherein Rₐ is a hydrolyzable group or a substituted or unsubstituted alkylene group interrupted with one or more hydrolyzable groups.

126. The method of claim 125, wherein the hydrolyzable group is an ester, a carbonate or a carbamate.

127. The method of claim 125, wherein each R₂ and R₃ are each independently an alkyl group or a hydroxyalkyl group.

128. The method of claim 127, wherein Q is represented by Structural Formula (III):

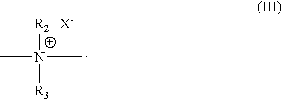

129. The method of claim 125, wherein Q is represented by the formula: 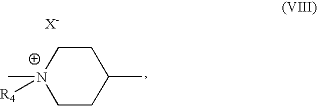
wherein R₄ is an electron pair, hydrogen or a substituted or unsubstituted lower alkyl group.

130. The method of claim 125, wherein Q is represented by the formula: 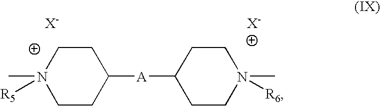
wherein A is a bond or substituted or unsubstituted lower alkylene group, and wherein R₅ and R₆ are each independently an electron pair, hydrogen or a substituted or unsubstituted lower alkyl group.

131. The method of claim 125, wherein Q is represented by the formula: 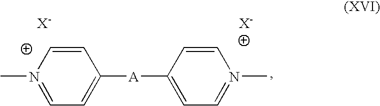
wherein A is a bond or a substituted or unsubstituted lower alkylene or lower alkylene glycol group.

132. The method of claim 131, wherein the repeat unit represented by Structural Formula (I) has the formula:

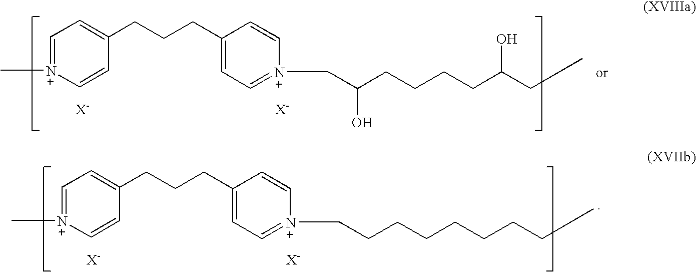

133. The method of claim 125, wherein repeat units represented by Structural Formula (I) have the formula: 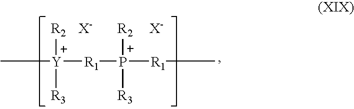
wherein:
Y is P or N;
R₁ is a substituted or unsubstituted hydrocarbyl group;
R₂ and R₃ are independently a substituted or unsubstituted aliphatic or aromatic group;
each X⁻ in the polymer or copolymer, separately or taken together with other X⁻s, is a physiologically acceptable anion.

134. The method of claim 125, wherein Q is represented by Structural Formula (VII): [chemical structure]
